(19) United States
(12) Patent Application Publication
(10) Pub. No.: US 2012/0095024 A1
(43) Pub. Date: Apr. 19, 2012

(54) TOLUIDINE SULFONAMIDES AND THEIR USE

(75) Inventors: Jorge Alonson, Frankfurt (DE); Arantxa Encionas Lopez, Karlsruhe (DE); Marcel Muelbaler, Marcel (DE); Lochen Ammenn, Sandhausen (DE); Bernd Vendt, Schondorf am Ammersee (DE); Joe Lewis, Dielhelm (DE); Christoph Schultes, Mannheim (DE); Bernd Janssen, Harvard, MA (US)

(73) Assignees: Elara Pharmaceuticals GmbH, Heidelberg (DE); European Molecular Biology Laboratory (EMBL), Heidelberg (DE)

(21) Appl. No.: 13/142,519

(22) PCT Filed: Dec. 30, 2009
(86) PCT No.: PCT/EP2009/009337

§ 371 Date: Dec. 23, 2011

(30) Foreign Application Priority Data
Dec. 30, 2008 (EP) PCT/EP2008/011147

(51) Int. Cl. A61K 31/505; C07D 213/64; C07D 213/84; C07D 239/28; C07D 333/38; A61K 31/41; A61K 31/4418; A61K 31/381; A61P 35/00; A61P 9/00; A61P 29/00; A61P 19/02; A61P 11/06; A61P 1/00; A61P 17/06; A61P 17/00; A61P 17/14; A61P 17/02; A61P 35/02; A61P 27/06; C07D 307/52; C07C 311/21; C07D 333/20; C07D 213/42; A61K 31/341; A61K 31/18; A61P 35/04; C07D 257/04

(52) U.S. Cl. 514/256; 548/252; 546/293; 546/286; 544/335; 549/61; 514/381; 514/357; 514/344; 514/438; 549/491; 564/92; 549/75; 546/338; 514/347; 514/471; 514/604

(57) ABSTRACT

The present invention provides novel compounds that inhibit cell proliferation and cell division and that inhibit the activation of Hypoxia Inducible Factor (HIF)-mediated transcription and signaling under hypoxic conditions. In one aspect, the compounds of the present invention are useful for the preparation of a medicament for the treatment or prevention of a disease or disorder selected from the group consisting of an inflammatory disease, a hyperproliferative disease or disorder, a hypoxia-related pathology and a disease characterized by excessive vascularisation. Also provided is a pharmaceutical composition comprising a compound of the invention and a second therapeutic agent or radiation useful for the treatment or prevention of the mentioned diseases or disorders. In a first aspect the present invention relates to a compound having a structure according to formula (I).

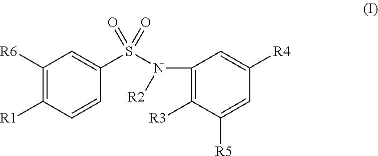

The present invention provides novel compounds that inhibit cell proliferation and cell division and that inhibit the activation of Hypoxia Inducible Factor (HIF)-mediated transcription and signaling under hypoxic conditions. In one aspect, the compounds of the present invention are useful for the preparation of a medicament for the treatment or prevention of a disease or disorder selected from the group consisting of an inflammatory disease, a hyperproliferative disease or disorder, a hypoxia-related pathology and a disease characterized by excessive vascularisation. Also provided is a pharmaceutical composition, comprising a compound of the invention and a second therapeutic agent or radiation, useful for the treatment or prevention of the mentioned diseases or disorders.

### BACKGROUND OF THE INVENTION

The normal response of cells to inadequate oxygen supply is mediated by the hypoxia signaling pathway. This response is important for a number of physiolocial functions such as tumor development and metastasis, resistance to apoptosis, induction of new blood vessel formation, and metabolism among others. For a general review on hypoxia signaling see e.g. Qingdong Ke and Max Costa, Molecular Pharmacology (2006), vol. 70, no. 5.

As a result of hypoxia, augmented levels of a heterodimeric complex of transcription factors (Hypoxia Inducible Factor, HIF), most notably HIF-1α and HIF-1β, are observed in e.g. tumors to compensate in cooperation with additional co-factors for the reduced availability of oxygen and nutrients in this fast growing tissue type. Under anaerobic conditions, homeostasis of HIF-1α is imbalanced by its reduced degradation, thus enabeling enhanced signaling through the Hypoxia Responsive Element (HRE) and resulting in increased expression of a large number of survival and growth factors.

Hypoxic conditions are also found in non-tumor tissue. For example, retinopathy is a general term that refers to non-inflammatory damage to the retina of the eye. This condition is most commonly caused by an insufficient blood supply leading to hypoxia. Particularly people with diabetes mellitus are at risk of retinopathy. The lack of oxygen in the retina of diabetics causes fragile, new, blood vessels to grow along the retina and in the clear, gel-like vitreous humour that fills the inside of the eye. Without timely treatment, these new blood vessels can bleed, cloud vision, and destroy the retina. Fibrovascular proliferation can also cause fractional retinal detachment. The new blood vessels can also grow into the anterior chamber of the eye and cause neovascular glaucoma.

Recently, evidence has accumulated that inhibition of HIF-1 activity could also act to prevent inflammation, by virtue of its role in the activation and infiltration of macrophages and neutrophils into affected tissues (see e.g. Giaccia et al., Drug Discovery, vol. 2, October 2003).

For the above-outlined reasons, compounds that inhibit HIF function are valuable medicaments for the treatment or prevention of a disease or disorder selected from the group consisting of an inflammatory disease, a hyperproliferative disease or disorder, a hypoxia-related pathology and a disease characterized by excessive vascularisation.

Because of the importance of HIF-1 in tumor development, progression and metastasis, a considerable amount of effort has been devoted to identify HIF-1 inhibitors for cancer therapy. A number of small molecules and RNA constructs, like siRNA, have been reported to exhibit inhibition of the HIF-1 pathway, e.g. Kung A L et al, Cancer Cell (2004), vol. 6, p. 33 ff; Rapisarda A, et al. Cancer Res. (2002), vol. 62, p. 4316 ff.; Tan C. et al, Cancer Res. (2005), vol. 65, p. 605 ff; Mabjeesh N J, et al, Cancer Cell, (2003), vol. 3, p. 363ff; Kong X, et al, Mol Cell Biol (2006), vol. 26, p. 2019 ff.; Kong D, et al, Cancer Res. (2005), vol. 65, p. 9047 ff.; Chau N. et al., Cancer Res. (2005), vol. 65, p. 4918 ff; Welsh S, et al., Mol Cancer Ther (2004), vol. 3, p. 233 ff. However, these compounds often have activities other than HIF-1 inhibition, and most of them lack the desired pharmacokinetic properties or toxicity profiles required for a useful pharmaceutical agent. Furthermore, some of the compounds have the disadvantage that they can not be administered orally, such as the HIF-1 inhibitor EZN-2968, which is a locked nucleic acid antisense oligonucleotide.

The scientific literature cited above emphasizes the high medical need for new therapeutic agents to provide more efficient treatment of different proliferative and inflammatory diseases or disorders, hypoxia-related pathologies and diseases characterized by excessive vascularisation.

### SUMMARY OF THE INVENTION

The present invention provides novelcompounds capable of prevention or treatment of a disease or disorder. Data presented herein establish that compounds according to the present invention are surprisingly very potent inhibitors of (i) the activation of HIF mediated transcription under hypoxic conditions, of (ii) cell cycle and cell proliferation, of (iii) estrogen response element—mediated transcriptional activity, and (iv) that these compounds induce apopotosis.

In a first aspect the present invention relates to a compound having a structure according to formula I:

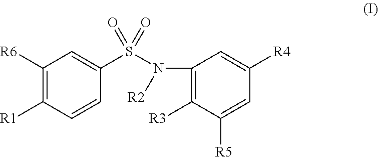

wherein
R¹ is selected from a group consisting of H, alkyl, alkenyl, alkynyl, —CN, halogen, —OH, alkoxy, —SH, S-alkyl, —NH₂, NH-alkyl, N-bis-alkyl, NHOH, NMeOH, NMe(OMe), —NO₂, —CF₃, —OCF₃ and C₁-C₄ hydroxyalkyl.
R² is H or C₁-C₄ alkyl;
R³ is H or —CH₃;
R⁴ is phenyl or monocyclic 5- or 6-membered heteroaryl; optionally substituted with one or more substituents selected from the group consisting of:
   alkyl, alkenyl, alkynyl, alkoxy, halogen, —CN, —CF₃, —OCF₃, C₁-C₄ hydroxyalkyl, —OH, —SH, S-alkyl, —CN, N-bis-alkyl, cyanoacetylene, —NO₂, —NR⁷R⁸, —C(O)R²⁰, N—O (wherein the nitrogen atom is integral part of the monocyclic 5- or 6-membered heteroaryl)
   and two substituents which form together a dioxymethylene bridge (—O—CH₂—o—);
R⁵ is H or —CH₃;
R⁶ is selected from the group consisting of H, halogen, alkyl, alkoxy, alkenyl, alkynyl, S-alkyl, —OH, —NR⁷R⁸, —CN, N-bis-alkyl, —SH, —CF₃ and —OCF₃; or R⁶ forms together with R¹ a dioxymethylene bridge (—O—CH₂—O—);
R⁷ is H or alkyl;
R⁸ is H or C₁-C₄ alkyl; and
R²⁰ is C₁-C₄ alkyl;
   with the proviso that R⁴ is not 3-alkoxy-pyridazine-5-yl; that if R⁴ is phenyl, then the 2- and 5-position of the phenyl ring may not be substituted with two methoxy substituents at the same time; and that R³ and R⁵ are not at the same time H.

In a further aspect the present invention relates to a pharmaceutical composition comprising a compound according to the invention or pharmaceutically acceptable salt thereof and a second therapeutic agent useful for the treatment or prevention of a disease or disorder selected from the group consisting of an inflammatory disease, a hyperproliferative disease or disorder, a hypoxia related pathology and a disease characterized by pathophysiological hyper-vascularisation, and, optionally, a pharmaceutically acceptable carrier or excipient.

In a further aspect the present invention is directed at the use of a compound according to the invention or the composition according to the invention for the preparation of a medicament for the treatment or prevention of a disease or disorder selected from the group consisting of an inflammatory disease, a hyperproliferative disease or disorder, a hypoxia related pathology and a disease characterized by pathophysiological hyper-vascularisation.

In another aspect, the invention provides a method for treating a hyperproliferative disease or disorder comprising administering a compound or composition according to the invention to a patient prior to, during and/or after he was subjected to a radiation therapy, a chemotherapy, an immunotherapy, a laser/microwave thermotherapy or a gene therapy using antisense DNA and RNA.

### DETAILED DESCRIPTION

It is to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the following definitions of the terms: alkyl, heteroalkyl, heteroaryl, alkenyl, and alkynyl are provided. These terms will in each instance of its use in the remainder of the specification have the respectively defined meaning and preferred meanings. Nevertheless in some instances of their use throughout the specification preferred meanings of these terms are indicated.

The term “alkyl” refers to a saturated straight or branched carbon chain. Preferably, the chain comprises from 1 to 10 carbon atoms, i.e. 1, 2, 3, 4, 5, 6, 7, 8, 9 or 10 carbon atoms, e.g. methyl, ethyl methyl, ethyl, propyl, iso-propyl, butyl, iso-butyl, tert-butyl, pentyl, hexyl, heptyl, octyl. Alkyl groups are optionally substituted where indicated.

The term “C₁-C₄ hydroxyalkyl” refers to a mono- or poly-hydroxylated C₁-C₄ alkyl, i.e. comprising 1, 2, 3 or 4 carbon atoms. Preferably, this term refers to a mono-hydroxylated C₁-C₄ alkyl group, e.g. hydroxymethyl, hydroxyethyl, hydroxypropyl, hydroxyl-iso-propyl, 1-hydroxybutyl or 2-hydroxybutyl.

The term “heteroalkyl” refers to a saturated straight or branched carbon chain. Preferably, the chain comprises from 1 to 9 carbon atoms, i.e. 1, 2, 3, 4, 5, 6, 7, 8, 9 carbon atoms, e.g. methyl, ethyl, propyl, iso-propyl, butyl, iso-butyl, sec-butyl, tert-butyl, pentyl, hexyl, heptyl, octyl, which is interrupted one or more times, e.g. 1, 2, 3, with the same or different heteroatoms. Preferably the heteroatoms are selected from O, S, and N, e.g. CH₂—O—CH₃, CH₂—O—C₂H₅, C₂H₄—O—CH₃, C₂H₄—O—C₂H₅ etc. Heteroalkyl groups are optionally substituted.

The term “heteroaryl” preferably refers to a five or six-membered aromatic monocyclic ring wherein at least one of the carbon atoms are replaced by 1, 2, 3 or 4 (for the five membered ring) or 1, 2, 3, or 4, (for the six membered ring) of the same or different heteroatoms, preferably selected from O, N and S; Examples are furanyl, thiophenyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl and 1,3,5-triazinyl.

As used herein, “monocyclic” refers to substituents having only one aromatic or heteroaromatic ring, excluding any multiple fused aromatic or heteroaromatic rings.

The terms “alkenyl” refer to olefinic unsaturated carbon atoms containing one or more double bonds. One example is propenyl. Preferably, the alkenyl chain comprises from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl, octenyl.

The term “alkynyl” refers to unsaturated carbon atoms with one or more triple bonds. An example is the propargyl radical. Preferably, the alkynyl chain comprises from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl.

In one preferred embodiment of the compounds of the invention, hydrogen atoms in an alkyl, aryl, alkenyl or alkynyl radical may be substituted independently from each other with one or more halogen atoms. One radical is the trifluoromethyl radical.

If two or more radicals can be selected independently from each other, then the term “independently” means that the radicals may be the same or may be different.

The term “pharmaceutically acceptable salt” refers to a salt of the compound of the present invention. Suitable pharmaceutically acceptable salts of the compound of the present invention include acid addition salts which may, for example, be formed by mixing a solution of the compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound of general formula (I)-(III). A prodrug is a pharmacologically active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters see Svensson and Tunek Drug Metabolism Reviews 16.5 (1988) and Bundgaard Design of Prodrugs, Elsevier (1985). Examples of a masked acidic anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 0 039 051 (Sloan and Little, Apr. 11, 1981) discloses Mannich-base hydroxamic acid prodrugs, their preparation and use.

Compounds of the present invention and also the starting materials for their preparation according to the invention can be synthesized as shown herein, and, alternatvely, by methods and standard procedures known to those skilled in the art, i.e. as described in the literature (for example in the standard works, such as Houben-Weyl, Methoden der organischen Chemie [Methods of Organic Chemistry], Georg-Thieme-Verlag, Stuttgart), under reaction conditions which are known to those skilled in the art and suitable for the said reactions.

If desired, the starting materials can also be formed in situ by not isolating them from the reaction mixture, but instead immediately converting them further into the compounds of the present invention. On the other hand, it is possible to carry out the reaction stepwise. It should be noted that the general procedures are shown as it relates to preparation of compounds having unspecified stereochemistry. However, such procedures are generally applicable to those compounds of a specific stereochemistry, e.g., where the stereochemistry at a sterogenic center is (S) or (R). In addition, the compounds having one stereochemistry (e.g., (R)) can often be utilized to produce those having opposite stereochemistry (i.e., (S)) using well-known methods, for example, by inversion.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds. The racemates, enantiomers, diastereomers, geometric isomers and individual isomers are all intended to be encompassed within the scope of the present invention. Accordingly, the compounds of this invention include mixtures of stereoisomers, especially mixtures of enantiomers, as well as purified stereoisomers, especially purified enantiomers, or stereoisomerically enriched mixtures, especially enantiomerically enriched mixtures. Also included within the scope of the invention are the individual isomers of the compounds represented by formulas (I) to (III) below as well as any wholly or partially equilibrated mixtures thereof. The present invention also covers the individual isomers of the compounds represented by the formulas below as mixtures with isomers thereof in which one or more chiral centers are inverted. Also, it is understood that all tautomers and mixtures of tautomers of the compounds of formulas (I) to (III) are included within the scope of the compounds of formulas (I) to (III) and preferably the formulas and subformulas corresponding thereto.

Racemates obtained can be resolved into the isomers mechanically or chemically by methods known per se. Diastereomers are preferably formed from the racemic mixture by reaction with an optically active resolving agent.

Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as—camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture.

The diastereomer resolution can also be carried out by standard purification processes, such as, for example, chromatography or fractional crystallization.

It is also possible to obtain optically active compounds of formulas (I) to (III) by the methods described above by using starting materials which are already optically active.

The present invention provides novel compositions, compounds and uses of these compounds and compositions for the prevention or treatment of a disease or disorder.

In a first aspect the present invention relates to a compound having a structure according to formula I:

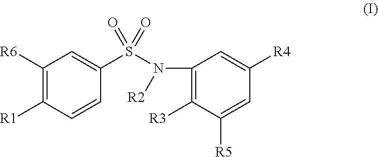

wherein
R¹ is selected from a group consisting of H; alkyl, preferably the alkyl chain comprises from 1 to 10 carbon atoms, i.e. 1, 2, 3, 4, 5, 6, 7, 8, 9 or 10 carbon atoms e.g. methyl, ethyl, propyl, iso-propyl, butyl, iso-butyl, tert-butyl, pentyl, hexyl, heptyl or octyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; —CN; halogen, in particular F, Cl, Br or I; —OH; alkoxy, in particular C₁-C₆ alkoxy, e.g. C₁, C₂, C₃, C₄, C₅, or C₆ alkoxy, preferably methoxy, ethoxy, propoxy, iso-propoxy, butoxy, iso-butoxy, tert-butoxy, pentoxy, or hexoxy; —SH; S-alkyl, in particular C₁-C₆ S-alkyl, e.g. C_{I}, C₂, C₃, C₄, C₅, or C₆ S-alkyl; —NH₂; NH-alkyl, in particular C₁-C₆ NH-alkyl, e.g. C₁, C₂, C₃, C₄, C₅, or C₆ NH-alkyl; N-bis-alkyl, preferably C₁-C₆ N-bis-alkyl, e.g. C₁, C₂, C₃, C₄, C₅, or C₆ N-bis-alkyl; NHOH; NMeOH; NMe(OMe); —NO₂; —CF₃; —OCF₃ and C₁-C₄ hydroxyalkyl, in particular C₁-C₄ hydroxyalkyl, e.g. C₁, C₂, C₃ or C₄ hydroxyalkyl, e.g. hydroxymethyl, hydroxyethyl, hydroxypropyl or hydroxyl-iso-propyl, 1-hydroxybutyl or 2-hydroxybutyl;
R² is H or C₁-C₄ alkyl, i.e. an alkyl group comprising from 1 to 4 carbon atoms, i.e. 1, 2, 3 or 4 carbon atoms e.g. methyl, ethyl, propyl, iso-propyl, butyl or iso-butyl;
R³ is H or —CH₃;
R⁴ is phenyl or monocyclic 5- or 6-membered heteroaryl; optionally substituted with one or more substituents selected from the group consisting of:
   alkyl, preferably the alkyl the chain comprises from 1 to 10 carbon atoms, i.e. 1, 2, 3, 4, 5, 6, 7, 8, 9 or 10 carbon atoms e.g. methyl, ethyl, propyl, iso-propyl, butyl, iso-butyl, tert-butyl, pentyl, hexyl, heptyl or octyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; alkoxy, in particular C₁-C₆ alkoxy, e.g. C₁, C₂, C₃, C₄, C₅, or C₆ alkoxy, preferably methoxy, ethoxy, propoxy, iso-propoxy, butoxy, iso-butoxy, tert-butoxy, pentoxy, or hexoxy; halogen, in particular F, Cl, Br or I; —CN; —CF₃; —OCF₃; C₁-C₄ hydroxyalkyl, in particular C₁-C₄ hydroxyalkyl, e.g. C₁, C₂, C₃ or C₄ hydroxyalkyl, e.g. hydroxymethyl, hydroxyethyl, hydroxypropyl or hydroxyl-iso-propyl, 1-hydroxybutyl or 2-hydroxybutyl; —OH; —SH; S-alkyl, in particular C₁-C₆ S-alkyl, e.g. C₁, C₂, C₃, C₄, C₅, or C₆ S-alkyl; —CN; N-bis-alkyl, preferably C₁-C₆N-bis-alkyl, e.g. C₁, C₂, C₃, C₄, C₅, or C₆ N-bis-alkyl; cyanoacetylene; —NO₂; —NR⁷R⁸, preferably —NH₂ or NH-alkyl, more preferably —NH₂ or —NH—C₁-C₄ alkyl, i.e. —NH—CH₃, —NH-ethyl, —NH—C₃ alkyl or —NH—C₄ alkyl and most preferably —NH₂, NH(CH₃) or N(CH₃)₂; —C(O)R²⁰, preferably acetyl, propionyl, iso-propionyl, butyryl or iso-butyryl; N-0 (wherein the nitrogen atom is integral part of the monocyclic 5- or 6-membered heteroaryl) and two substituents which form together a dioxymethylene bridge (—O—CH₂—O—);
R⁵ is H or —CH₃;
R⁶ is selected from the group consisting of H; halogen, in particular F, Cl, Br or I; alkyl, preferably the alkyl the chain comprises from 1 to 10 carbon atoms, i.e. 1, 2, 3, 4, 5, 6, 7, 8, 9 or 10 carbon atoms e.g. methyl, ethyl, propyl, iso-propyl, butyl, iso-butyl, tert-butyl, pentyl, hexyl, heptyl or octyl; alkoxy, in particular C₁-C₆ alkoxy, e.g. C₁, C₂, C₃, C₄, C₅, or C₆ alkoxy, preferably methoxy, ethoxy, propoxy, iso-propoxy, butoxy, iso-butoxy, tert-butoxy, pentoxy, or hexoxy; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; S-alkyl, in particular C₁-C₆ S-alkyl, e.g. C₁, C₂, C₃, C₄, C₅, or C₆ S-alkyl; —OH; —NR⁷R⁸, preferably —NH₂ or NH-alkyl, more preferably —NH₂ or —NH—C₁-C₄ alkyl, i.e. —NH—CH₃, —NH-ethyl, —NH—C₃ alkyl or —NH—C₄ alkyl and most preferably —NH₂, NH(CH₃) or N(CH₃)₂; —CN; N-bis-alkyl, preferably C₁-C₆ N-bis-alkyl, e.g. C₁, C₂, C₃, C₄, C₅, or C₆ N-bis-alkyl; —SH; —CF₃ and —OCF₃;
   or R⁶ forms together with R¹ a dioxymethylene bridge (—O—CH₂—O—), optionally substituted at the methylene;
R⁷ is H or alkyl, preferably the alkyl the chain comprises from 1 to 10 carbon atoms, i.e. 1, 2, 3, 4, 5, 6, 7, 8, 9 or 10 carbon atoms e.g. methyl, ethyl, propyl, iso-propyl, butyl, iso-butyl, tert-butyl, pentyl, hexyl, heptyl or octyl;
R⁸ is H or C₁-C₄ alkyl, in particular methyl, ethyl, propyl, iso-propyl, butyl or iso-butyl; and
R²⁰ is C₁-C₄ alkyl, in particular methyl, ethyl, propyl, iso-propyl, butyl or iso-butyl;
   with the proviso that R⁴ is not 3-alkoxy-pyridazine-5-yl; if R⁴ is phenyl, then the 2- and 5-position of the phenyl ring may not be substituted with two methoxy substituents at the same time; and that R³ and R⁵ are not at the same time H.

Also preferred is a compound, wherein R¹ is selected from the group consisting of —CH₃, —OCH₃, —CF₃, F and —NH₂.

In a further preferred embodiment of the compound, R⁶, R² and R⁵ is H and R¹ is selected from the group consisting of —CH₃, —OCH₃, —CF₃, F and —NH₂.

The R⁴ substituent of a compound of the invention may have in preferred embodiments only a single heteroatom and/or may be substituted with less than two or less than three substituents.

A further preferred embodiment of the compound of the invention has the following substituents: R⁶, R² and R⁵ is H and R¹ is selected from the group consisting of —CH₃, —OCH₃, —CF₃, F and —NH₂ and the R⁴ group is substituted with less than two or less than three substituents and/or, if R⁴ is a monocyclic 5- or 6-membered heteroaryl, then it is preferred in this context that it comprises only a single heteroatom.

In a preferred embodiment the compound of the invention has a structure according to formula II:

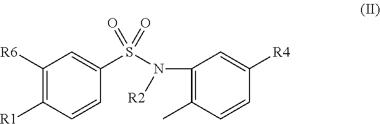

Also preferred is a compound of the invention having a structure according to formula III:

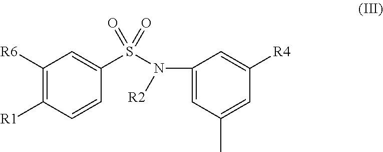

In the context of formulas (II) and (III) R¹, R², R⁴ and R⁶ have the above indicated meaning and preferred meanings. Particularly preferred is a compound of formula (II) or (III), wherein R¹ is selected from the group consisting of —CH₃, —OCH₃, —CF₃, F and —NH₂. In a further preferred embodiment of the compound according to formula (II) or (III) R² is H. In an even more preferred embodiment R⁶ and R² are H and R¹ is selected from the group consisting of —CH₃, —OCH₃, —CF₃, F and —NH₂.

In a preferred embodiment of the compound of the invention, preferably a compound according to formula (II) or (III), R⁴ has a structure according to formula IV:

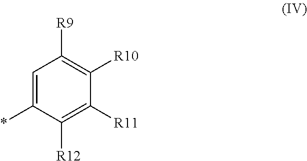

wherein
R⁹ and R¹⁰ are each individually selected from the group consisting of H; C₁-C₄ alkyl, in particular methyl, ethyl, propyl, iso-propyl, butyl or iso-butyl; C₁-C₄ alkenyl, e.g. ethenyl, 1-propenyl, 2-propenyl, iso-propenyl, 1-butenyl, 2-butenyl, 3-butenyl, iso-butenyl or sec-butenyl; C₁-C₄ alkynyl, e.g. ethynyl, 1-propynyl, 2-propynyl, 1-butynyl, 2-butynyl or 3-butynyl; —CN; —C(O)R²⁰, preferably acetyl, propionyl, iso-propionyl, butyryl or iso-butyryl; cyanoacetylene; halogen, in particular F, Cl, Br or I; —OH; C₁-C₄ alkoxy, e.g. methoxy, ethoxy, propoxy, iso-propoxy, butoxy, iso-butoxy or tert-butoxy; —SH; C₁-C₄ S-alkyl, e.g. C₁, C₂, C₃ or C₄ S-alkyl; —NH₂; C₁-C₄ NH-alkyl, e.g. C₁, C₂, C₃ or C₄ NH-alkyl; C₁-C₄ N-bis-alkyl, e.g. C₁, C₂, C₃ or C₄ N-bis-alkyl; —NO₂; —CF₃; —OCF₃; and C₁-C₄ hydroxyalkyl, e.g. hydroxymethyl, hydroxyethyl, hydroxypropyl or hydroxyl-iso-propyl, 1-hydroxybutyl or 2-hydroxybutyl; or R⁹ and R¹⁰ form together a dioxymethylene bridge (—O—CH₂—O—);
R¹¹ and R¹² are each individually selected from the group consisting of H; C₁-C₄ alkyl in particular methyl, ethyl, propyl, iso-propyl, butyl or iso-butyl; C₁-C₄ alkenyl, e.g. ethenyl, 1-propenyl, 2-propenyl, iso-propenyl, 1-butenyl, 2-butenyl, 3-butenyl, iso-butenyl or sec-butenyl; C₁-C₄ alkynyl, e.g. ethynyl, 1-propynyl, 2-propynyl, 1-butynyl, 2-butynyl or 3-butynyl; —CN; halogen, in particular F, Cl, Br or I; —OH; C₁-C₄ alkoxy, e.g. methoxy, ethoxy, propoxy, iso-propoxy, butoxy, iso-butoxy or tert-butoxy; —SH; C₁-C₄ S-alkyl, e.g. C₁, C₂, C₃ or C₄ S-alkyl; —CF₃; —OCF₃; —NH₂; —N(CH₃)₂ and C₁-C₄ hydroxyalkyl, e.g. hydroxymethyl, hydroxyethyl, hydroxypropyl or hydroxyl-iso-propyl, 1-hydroxybutyl or 2-hydroxybutyl;
   with the proviso that R⁹ and R¹² may not be methoxy at the same time;
R²⁰ has the above indicated meaning, e.g. methyl, ethyl, propyl, iso-propyl, butyl or iso-butyl; and
* indicates the bond between R⁴ and the compound according to any of formulas (I) through (III).

In the preferred embodiment wherein R⁴ has the structure of formula (IV) it is particularly preferred that (a) R⁹ is CN, R¹⁰, R¹¹ and R¹² is H; (b) R¹⁰ is CN and R⁹, R¹¹ and R¹² is H, (c) R⁹, R¹⁰ and R¹¹ is H and R¹² is CN; (d) R⁹ is ethynyl, R¹⁰, R¹¹ and R¹² is H; (e) R¹⁰ is ethynyl, R⁹, R¹¹ and R¹² is H, (f) R⁹, R¹⁰ and R¹¹ is H and R¹² is ethynyl; (g) R⁹ is CN and one of R¹⁰, R¹¹ and R¹² is OH or C₁-C₄ alkoxy, e.g. methoxy, ethoxy, propoxy, iso-propoxy, butoxy, iso-butoxy or tert-butoxy; (h) R¹⁰ is CN and one of R⁹, R¹¹ and R¹², preferably R⁹ is halogen, in particular F, OH or C₁-C₄ alkoxy, e.g. methoxy, ethoxy, propoxy, iso-propoxy, butoxy, iso-butoxy or tert-butoxy or (i) one of R⁹, R¹⁰ and R¹¹ is OH or C₁-C₄ alkoxy, e.g. methoxy, ethoxy, propoxy, iso-propoxy, butoxy, iso-butoxy or tert-butoxy and R¹² is CN; preferably R¹⁰ is CN and R⁹ is OH or C₁-C₄ alkoxy, preferably methoxy.

Further preferred is a compound of the invention, wherein R¹¹ and R¹² are H.

In another preferred embodiment of the compound of the invention, R³ is methyl; R² is H, methyl or ethyl, preferably H; and R⁵ and R⁶ are H.

In a preferred embodiment of the compound of the invention, R⁴ has a structure according to formula V:

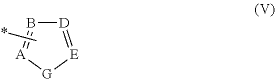

wherein
A, B, D and E are each individually selected from the group consisting of a nitrogen atom, CR¹³ and N—O;
G is selected from the group consisting of an oxygen atom, a sulphur atom and NR¹⁴;
R¹³ is selected from the group consisting of H; C₁-C₃ alkyl, in particular methyl, ethyl, propyl or iso-propyl; C₁-C₃ alkoxy, e.g. methoxy, ethoxy, propoxy or iso-propoxy; —OH; —SH; S-alkyl, e.g. C₁, C₂ or C₃ S-alkyl; —CF₃; —OCF₃; halogen, in particular F, Cl, Br or I; —NR¹⁵R¹⁶, preferably —NH₂ or —NH—C₁-C₄ alkyl, i.e. —NH—CH₃, —NH-ethyl, —NH—C₃ alkyl or —NH—C₄ alkyl and most preferably —NH₂, NH(CH₃) or N(CH₃)₂; —NO₂; —CN; —C(O)R²⁰; acetylene; cyanoacetylene; C₁-C₄ hydroxyalkyl, e.g. hydroxymethyl, hydroxyethyl, hydroxypropyl or hydroxyl-iso-propyl, 1-hydroxybutyl or 2-hydroxybutyl and a σ (sigma) bond connecting R⁴ to the compound according to any of formulas (I) through (III); and
R¹⁴ is selected from the group consisting of H, C₁-C₄ alkyl, in particular methyl, ethyl, propyl, iso-propyl, butyl or iso-butyl and more preferably methyl, ethyl, propyl or iso-propyl; and a σ (sigma) bond connecting R⁴ to the compound according to any of formulas (I) through (III); and
R¹⁵ and R¹⁶ are each individually either H or C₁-C₄ alkyl, in particular methyl, ethyl, propyl, iso-propyl, butyl or iso-butyl;
R²⁰ has the above indicated meaning, e.g. methyl, ethyl, propyl, iso-propyl, butyl or iso-butyl; and
* indicates the bond between R⁴ and the compound according to any of formulas (I) through (III).

In another preferred embodiment of the compound of the invention, R⁴ has a structure according to formula VI:

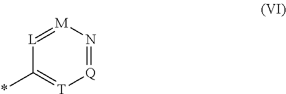

wherein
L and T are each individually either a CH group or a nitrogen atom or N—O;
M, N and Q are each individually selected from the group consisting of a nitrogen atom, a CR¹⁷ group and N—O;
R¹⁷ is selected from the group consisting of H; C₁-C₃ alkyl, in particular methyl, ethyl, propyl or iso-propyl; C₁-C₃ alkoxy, e.g. methoxy, ethoxy, propoxy or iso-propoxy; —CF₃; —OCF₃; halogen, in particular F, Cl, Br or I; —OH; —NO₂; —SH; C₁-C₃ S-alkyl, e.g. C₁, C₂ or C₃ S-alkyl; —NR¹⁵R¹⁶, preferably —NH₂ or —NH—C₁-C₄ alkyl, i.e. —NH—CH₃, —NH-ethyl, —NH—C₃ alkyl or —NH—C₄ alkyl and most preferably —NH₂, NH(CH₃) or N(CH₃)₂; C₁-C₄ hydroxyalkyl, e.g. hydroxymethyl, hydroxyethyl, hydroxypropyl or hydroxyl-iso-propyl, 1-hydroxybutyl or 2-hydroxybutyl; —C(O)R²⁰, preferably acetyl, propionyl, iso-propionyl, butyryl or iso-butyryl; acetylene; cyanoacetylene and —CN;
R¹⁵ and R¹⁶ are each individually either H or C₁-C₄ alkyl;
R²⁰ has the above indicated meaning, e.g. methyl, ethyl, propyl, iso-propyl, butyl or iso-butyl; and
* indicates the bond between R⁴ and the compound according to any of formulas (I) through (III).

Also preferred is a compound of the invention, wherein R⁴ is selected from the group consisting of:

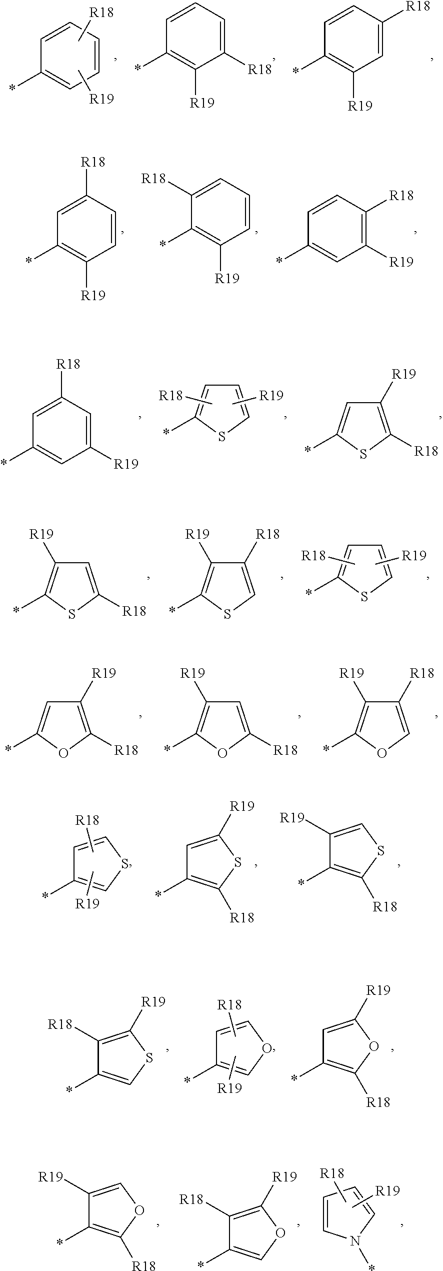

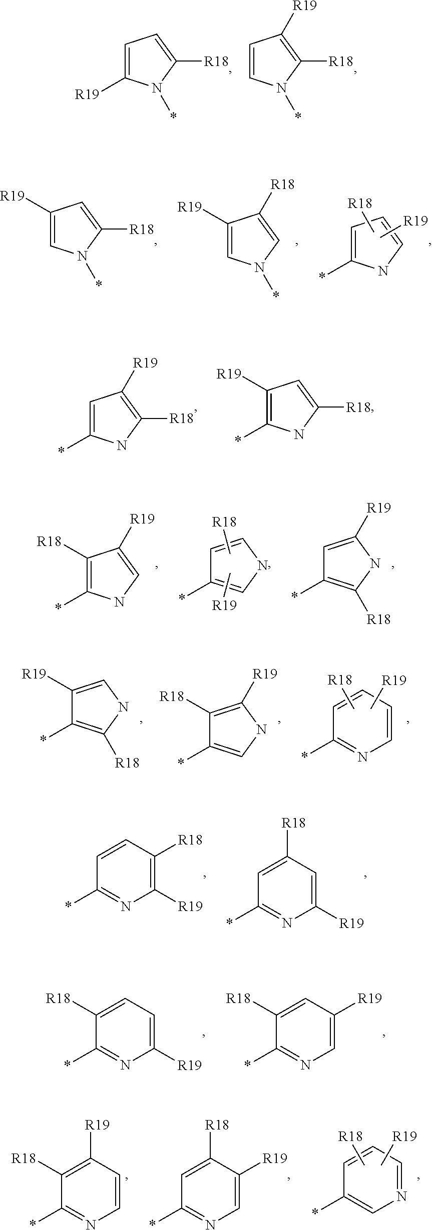

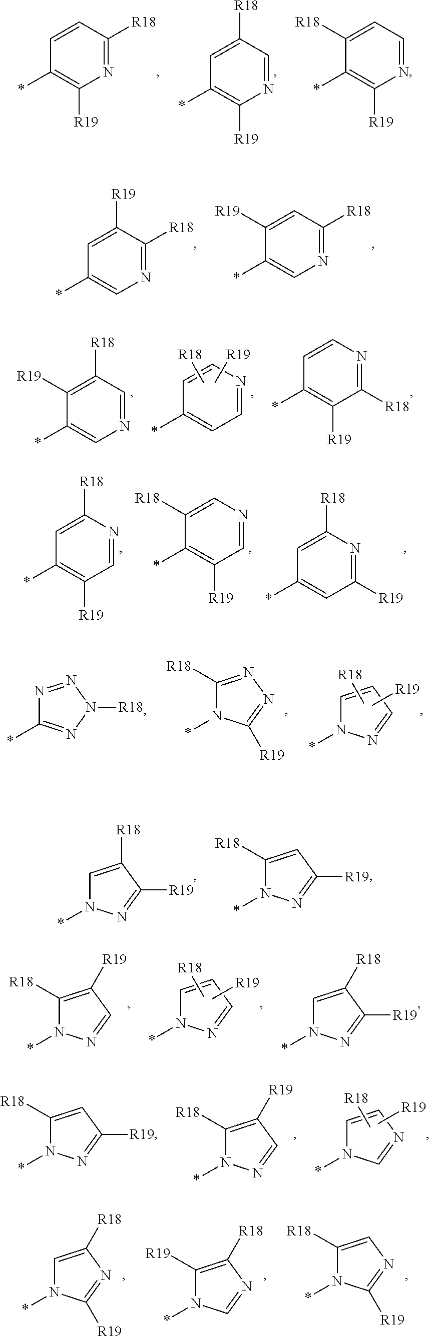

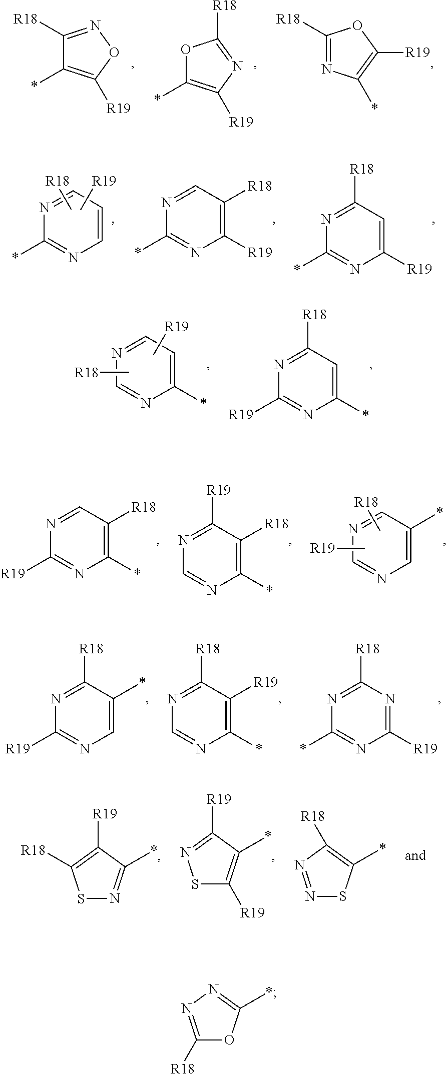

wherein R¹⁸ and R¹⁹ are each individually selected from the group consisting of H; C₁-C₃ alkyl, in particular methyl, ethyl, propyl or iso-propyl; C₁-C₃ alkoxy, e.g. methoxy, ethoxy, propoxy or iso-propoxy; —CF₃; —OCF₃; halogen, in particular F, Cl, Br or I; —OH; —NO₂; —SH; C₁-C₃ S-alkyl, e.g. C₁, C₂ or C₃ S-alkyl; —NR¹⁵R¹⁶, preferably —NH₂ or —NH—C₁-C₄ alkyl, i.e. —NH—CH₃, —NH-ethyl, —NH—C₃-alkyl or —NH—C₄-alkyl and most preferably —NH₂, NH(CH₃) or N(CH₃)₂; C₁-C₄-hydroxyalkyl, e.g. hydroxymethyl, hydroxyethyl, hydroxypropyl or hydroxyl-iso-propyl, 1-hydroxybutyl or 2-hydroxybutyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; —C(O)R⁶ preferably acetyl, propionyl, iso-propionyl, butyryl or iso-butyryl; cyanoacetylene and —CN;
and R¹⁵ and R¹⁶ have the above indicated meaning and preferred meaning.

In a preferred embodiment R¹⁸ is H and R¹⁹ in above indicated structures is selected from the group consisting of C₁-C₃ alkyl, in particular methyl, ethyl, propyl or iso-propyl; C₁-C₃ alkoxy, e.g. methoxy, ethoxy, propoxy or iso-propoxy; —CF₃; —OCF₃; halogen, in particular F, Cl, Br or I; —OH; —NO₂; —SH; C₁-C₃ S-alkyl, e.g. C₁, C₂ or C₃ S-alkyl; —NR¹⁵R¹⁶, preferably —NH₂ or —NH—C₁-C₄ alkyl, i.e. —NH—CH₃, —NH-ethyl, —NH—C₃-alkyl or —NH—C₄-alkyl and most preferably —NH₂, NH(CH₃) or N(CH₃)₂; C₁-C₄-hydroxyalkyl, e.g. hydroxymethyl, hydroxyethyl, hydroxypropyl or hydroxyl-iso-propyl, 1-hydroxybutyl or 2-hydroxybutyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; —C(O)R⁶ preferably acetyl, propionyl, iso-propionyl, butyryl or iso-butyryl; cyanoacetylene and —CN. The most preferred meanings of R¹⁹ in this context are C₁-C₃ alkyl, in particular methyl, ethyl, propyl or iso-propyl; C₁-C₃ alkoxy, in particular methoxy, ethoxy, propoxy or iso-propoxy; —CF₃; —OCF₃, —CN, —NO₂, or halogen, in particular F, Cl, Br or I, with methyl, methoxy, CF₃ or —CN being the most preferred meanings.

In another preferred embodiment R¹⁹ is H and R¹⁸ in above indicated structures is selected from the group consisting of C₁-C₃ alkyl, in particular methyl, ethyl, propyl or iso-propyl; C₁-C₃ alkoxy, e.g. methoxy, ethoxy, propoxy or iso-propoxy; —CF₃; —OCF₃; halogen, in particular F, Cl, Br or I; —OH; —NO₂; —SH; C₁-C₃ S-alkyl, e.g. C₁, C₂ or C₃ S-alkyl; —NR¹⁵R¹⁶, preferably —NH₂ or —NH—C₁-C₄ alkyl, i.e. —NH—CH₃, —NH-ethyl, —NH—C₃-alkyl or —NH—C₄-alkyl and most preferably —NH₂, NH(CH₃) or N(CH₃)₂; C₁-C₄-hydroxyalkyl, e.g. hydroxymethyl, hydroxyethyl, hydroxypropyl or hydroxyl-iso-propyl, 1-hydroxybutyl or 2-hydroxybutyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; —C(O)R⁶ preferably acetyl, propionyl, iso-propionyl, butyryl or iso-butyryl; cyanoacetylene and —CN. The most preferred meanings of R¹⁸ in this context are C₁-C₃ alkyl, in particular methyl, ethyl, propyl or iso-propyl; C₁-C₃ alkoxy, in particular methoxy, ethoxy, propoxy or iso-propoxy; —CF₃; —OCF₃, —CN, NO₂, or halogen, in particular F, Cl, Br or I, with methyl, methoxy, CF₃ or —CN being the most preferred meanings.

In another preferred embodiment R¹⁸ and R¹⁹ are each individually selected from the group consisting of C₁-C₃ alkyl, in particular methyl, ethyl, propyl or iso-propyl; C₁-C₃ alkoxy, e.g. methoxy, ethoxy, propoxy or iso-propoxy; —CF₃; —OCF₃; halogen, in particular F, Cl, Br or I; —OH; —NO₂; —SH; C₁-C₃ S-alkyl, e.g. C₁, C₂ or C₃ S-alkyl; —NR¹⁵R¹⁶, preferably —NH₂ or —NH—C₁-C₄ alkyl, i.e. —NH—CH₃, —NH-ethyl, —NH—C₃-alkyl or —NH—C₄-alkyl and most preferably —NH₂, NH(CH₃) or N(CH₃)₂; C₁-C₄-hydroxyalkyl, e.g. hydroxymethyl, hydroxyethyl, hydroxypropyl or hydroxyl-iso-propyl, 1-hydroxybutyl or 2-hydroxybutyl; alkenyl, particularly an alkenyl chain comprising from 2 to 8 carbon atoms, i.e. 2, 3, 4, 5, 6, 7, or 8 carbon atoms, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl or octenyl; alkynyl, preferably an alkynyl comprising from 2 to 8 carbon atoms, i.e. 2, 3, 4, 5, 6, 7, or 8 carbon atoms, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl or octynyl; —C(O)R⁶ preferably acetyl, propionyl, iso-propionyl, butyryl or iso-butyryl; cyanoacetylene and —CN. The most preferred meanings of R¹⁸ and R¹⁹ in this context are C₁-C₃ alkyl, in particular methyl, ethyl, propyl or iso-propyl; C₁-C₃ alkoxy, in particular methoxy, ethoxy, propoxy or iso-propoxy; —CF₃; —OCF₃, —CN, NO₂, or halogen, in particular F, Cl, Br or I, with methyl, methoxy, CF₃ or —CN being the most preferred meanings.

In another preferred embodiment of the compound of the invention, R² is H and R⁴ is selected from the group consisting of:

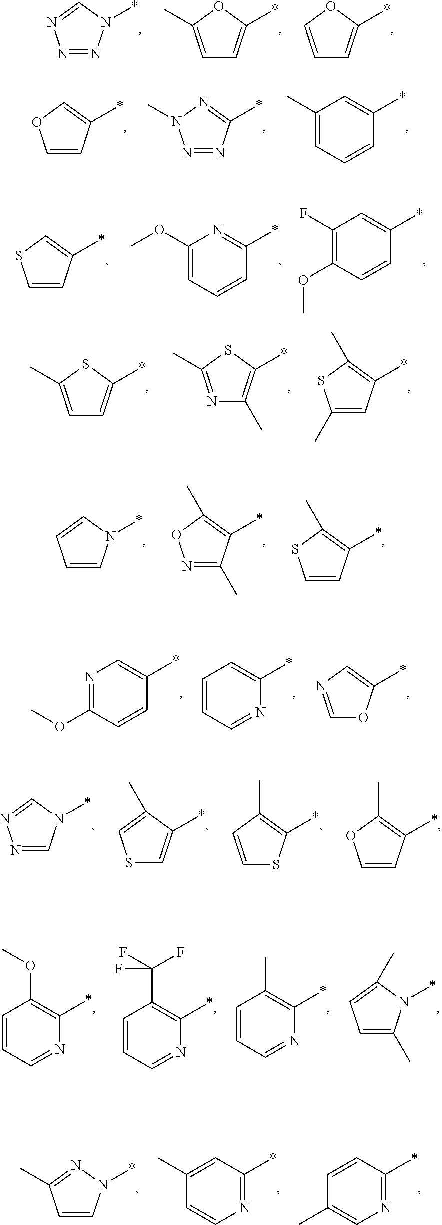

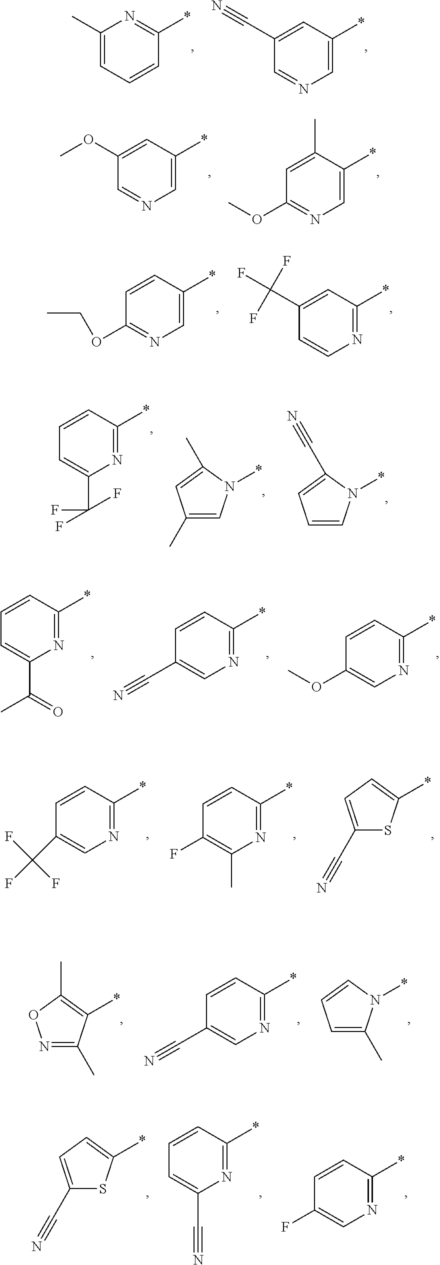

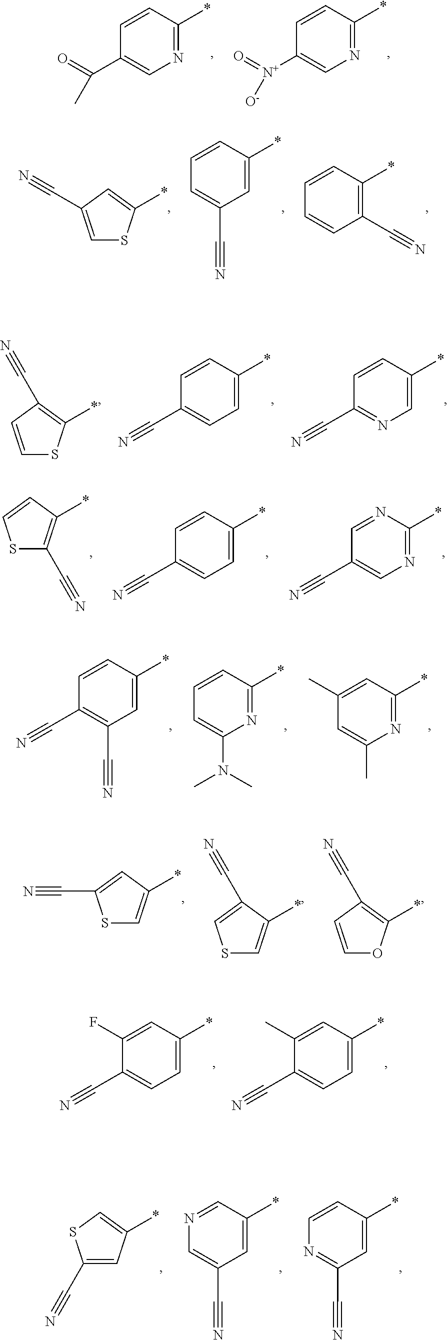

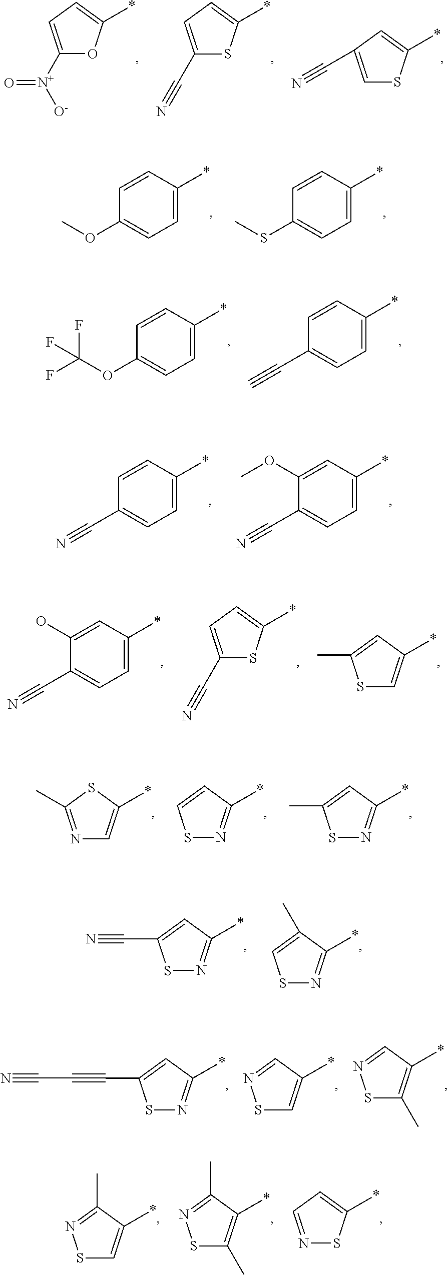

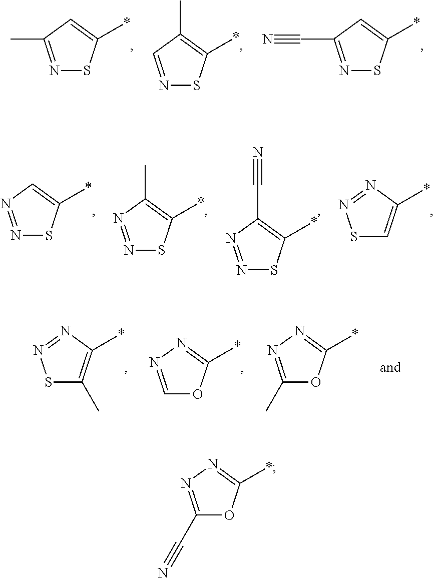

wherein * indicates the bond between R⁴ and the compound according to any of formulas (I) through (III).

In a further preferred embodiment of the compound of the invention R² and/or R⁶ is H.

The following Table 1 lists further preferred combinations of specific substituents of the compound of the invention:

TABLE 1

| R¹ | R² | R³ | R⁴ | R⁵ | R⁶ | R¹¹ | R¹² |
|---|---|---|---|---|---|---|---|
| as defined | H | methyl | as defined | H | H | as | as |
| herein | | | herein | | | defined | defined |
| | | | | | | herein | herein |
| as defined | H | methyl | as defined | H | as defined | as | as |
| herein | | | herein | | herein | defined | defined |
| | | | | | | herein | herein |
| as defined | H | as defined | as defined | as defined | H | as | as |
| herein | | herein | herein | herein | | defined | defined |
| | | | | | | herein | herein |
| as defined | H | as defined | as defined | as defined | as defined | as | as |
| herein | | herein | herein | herein | herein | defined | defined |
| | | | | | | herein | herein |
| as defined | as defined | as defined | as defined | as defined | H | as | as |
| herein | herein | herein | herein | herein | | defined | defined |
| | | | | | | herein | herein |
| as defined | H | methyl | as defined | H | H | H | H |
| herein | | | herein | | | | |
| as defined | H | methyl | as defined | H | as defined | H | H |
| herein | | | herein | | herein | | |
| as defined | H | as defined | as defined | as defined | H | H | H |
| herein | | herein | herein | herein | | | |
| as defined | H | as defined | as defined | as defined | as defined | H | H |
| herein | | herein | herein | herein | herein | | |
| as defined | as defined | as defined | as defined | as defined | H | H | H |
| herein | herein | herein | herein | herein | | | |

In a particularly preferred embodiment of the compound of the invention, the compound is selected from the group of compounds listed in Table 2A or Table 7.

In a further aspect the invention provides a compound according to the invention or pharmaceutically acceptable salt thereof, for the prevention or treatment of a disease or disorder.

As shown in the examples below, the advantageous properties of the compounds of the invention include their ability of effectively inhibiting cell proliferation and their activity as HIF inhibitors. For example, the compounds of the present invention were shown to inhibit the activation of HIF-mediated transcription under hypoxic conditions. Thus, the compounds of the invention can be used for the preparation of a medicament for the treatment of a disorder characterized by pathophysiological HIF signaling. A person skilled in the art of medical, biological and/or pharmacological science can determine with routine methodology if a disorder is characterized by undesirable HIF signaling. Tissues affected by such diseases will overexpress genes that are induced by activation of the HIF responsive element (HRE). HIF-1 acts by binding to HIF-responsive elements (HREs) in promoters that generally contain the sequence NCGTG. The genes affected by HIF activity which are regulated by said promoters are well known in the art and were also described in multiple reviews (see e.g. FIG. 3 of Gregg L. Semenza, Nature Reviews, October 2003, vol. 3).

In animal studies, HIF-1 overexpression is associated with increased tumor growth, increased vascularisation, metastasis and fibrosis, e.g. renal fibrosis (see: Semenza, G, Drug Discovery Today, vol. 12, no. 19/20, October 2007; Kimura, Kuniko, et al., American Journal of Physiology (2008), 295(4, Pt. 2), F1023-F1029 and for a review see N. J. Mabjeesh et al., Histol. Histopathol (2007) 22:559-572). Fibrosis is the formation or development of excess fibrous connective tissue in an organ or tissue. Recently, it has become clear that inhibition of HIF-1 activity also acts to prevent inflammation, by virtue of its essential role in the activation and infiltration of macrophages and neutrophils into affected tissues (see e.g. Giaccia et al., Drug Discovery, vol. 2, October 2003).

### Pharmaceutical Compositions

For the above mentioned reasons, a compound of the present invention can be used to treat an inflammatory disease, a hyperproliferative disease or disorder, a hypoxia related pathology and also diseases characterized by pathophysiological hyper-vascularisation. Therefore, as a further aspect, the invention provides a therapeutical composition wherein the compound of the invention is combined with at least one further pharmaceutically active compound that is useful to treat one of the aforementioned diseases or disorders. Such therapeutical compositions are useful because the therapeutic efficiency of the compounds of the invention can be amplified by the presence of said at least one further pharmaceutically active compound and vice versa. For example, it was shown that inhibiting HIF1α activity via antisense gene therapy enhances the therapeutic efficacy of doxorubicin to combat hepatocellular carcinoma (see Liu, Fengjun et. al., Cancer Science (2008), 99(10), 2055-2061).

Thus, in a further aspect, the present invention relates to a pharmaceutical composition comprising a compound according to the invention or pharmaceutically acceptable salt thereof and a second therapeutic agent useful for the treatment or prevention of a disease or disorder selected from the group consisting of an inflammatory disease, a hyperproliferative disease or disorder, a hypoxia related pathology and a disease characterized by pathophysiological hyper-vascularisation, and, optionally, a pharmaceutically acceptable carrier or excipient. Such compositions are also useful to obtain synergistic therapeutic effects and also to prevent drug resistance of tumor cells, for example. It is also for these reasons, that current chemotherapy generally involves administering a cocktail of different cytotoxic and/or cytostatic compounds to improve the effectiveness of the treatment and reduce the possibility of tumor cell adaptation. In a further aspect, the present invention relates to a pharmaceutical composition comprising a compound according to the invention or pharmaceutically acceptable salt thereof in combination with radiation therapies.

Any composition of the present invention may be admixed with a pharmaceutically acceptable diluent, excipient or carrier, or a mixture thereof.

Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients”, 2nd Edition, (1994), Edited by A Wade and P J Weller. Acceptable carriers or diluents for therapeutic use are well known in the pharmaceutical art, and are described, for example, in Remington's Pharmaceutical Sciences, Mack Publishing Co. (A. R Gennaro edit. 1985).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 80%, more preferably from 20% to 70% of the active compound or active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. Liquid forms are particularly preferred for topical applications to the eye. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Interestingly, HIF inhibitors, such as the compounds of the invention, can prevent the development of tumor resistance towards chemotherapeutic drugs and can make cancer cells more sensitive towards radiotherapy (see e.g. Palayoor S T, et al., Int J Cancer. 2008 Nov. 15; 123(10):2430-7 and Gregg L. Semenza, Nature Reviews, October 2003, vol. 3). Thus, useful second therapeutic agents that can be combined with a compound of the invention to produce the pharmaceutical composition of the invention include, without limitation, a (further) HIF-1 inhibitor, a cytotoxic compound and cytostatic compounds.

A HIF-1 inhibitor can be, e.g. selected from the group consisting of PX-478 (S-2-amino-3-[4′-N,N,-bis(2-chloroethyl)amino]phenyl propionic acid N-oxide dihydrochloride); a topoisomerase-1 inhibitor such as 8,9-Dimethoxy-5-(2-N,N-dimethylaminoethyl)-2,3-methylenedioxy-5H-dibenzo[c,h][1,6]naphthyridin-6-one (also known as ARC-111 or topovale) or (S)-10-[(dimethylamino)methyl]-4-ethyl-4,9-dihydroxy-1H-pyrano[3′,4′:6,7]indolizino[1, 2-1)]quinoline-3,14-(4H, 12H)-dione monohydrochloride (also referred to as tropotecan); echinomycin; chetomin (NSC289491); cyclosporine A; 3-[2-[4-[bis(4-fluorophenyl)methylene]-1-piperidinyl]-2,3-dihydro-2thioxo-4(1H)-quinazolinone (R59949); an inhibitor of the PIK3K/Akt/mTor signalling cascade, e.g., LY294002, wortmannin or rapamycin; an inhibitor of the MAPK signalling cascade, e.g. the MEK1 inhibitor PD98059; a soluble guanyl cyclase stimulator such as 3-(5′ hydroxymethyl-2′-furyl)-1-benzylindazole (YC-1); a heat-shock protein 90 inhibitor, in particular radicicol, the radicicol analogue KF58333 or geldanamycin; a microtubule disrupting agent, in particular e.g. taxol, vincristine or 2-methoxyestradiol; a histone deacetylase inhibitor, e.g. FK228; a thioredoxin inhibitor, in particular PX-12 or pleurotin; UCNO-1; diphenylene iodonium, genestein and carboxyamido-triazole.

Many cytotoxic or cytostatic compounds are known to the expert artisan skilled in the therapy of hyperproliferative diseases or disorders such as a tumor or cancer disease. For example, cytotoxic and cytostatic compounds include, but are not limited to, pure or mixed anti-estrogens such as faslodex, tamoxifen or raloxifen; any inhibitors of topoisomerase I or II, such as camptothecin (topo I) or etoposide (topo II); any compound that acts through inhibiting aromatase activity, such as anastrozole or letrozole; any preparation that interferes with HER2 signalling such as herceptin; any compound that interchelates DNA, such as doxorubicin. Particularly preferred cytostatic or cytotoxic drugs, which can be combined with the compounds of the present invention are alkylating substances, anti-metabolites, antibiotics, epothilones, nuclear receptor agonists and antagonists, anti-androgenes, anti-estrogens, platinum compounds, hormones and antihormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneimenes, methylmelamines, trazines, vinca alkaloids, pyrimidine analogs, purine analogs, alkylsulfonates, folic acid analogs, anthracendiones, substituted urea, methylhydrazin derivatives, in particular acediasulfone, aclarubicine, ambazone, aminoglutethimide, L-asparaginase, azathioprine, bleomycin, busulfan, calcium folinate, carboplatin, carpecitabine, carmustine, celecoxib, chlorambucil, cis-platin, cladribine, cyclophosphamide, cytarabine, dacarbazine, dactinomycin dapsone, daunorubicin, dibrompropamidine, diethylstilbestrole, docetaxel, doxorubicin, enediynes, epirubicin, epothilone B, epothilone D, estramucin phosphate, estrogen, ethinylestradiole, etoposide, flavopiridol, floxuridine, fludarabine, fluorouracil, fluoxymesterone, flutamide fosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon γ, irinotecan, leuprolide, lomustine, lurtotecan, mafenide sulfate olamide, mechlorethamine, medroxyprogesterone acetate, megastrolacetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, scriflavinium chloride, semustine streptozocine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, timidazole, topotecan, triaziquone, treosulfan, trimethoprim, trofosfamide, UCN-01, vinblastine, vincristine, vindesine, vinblastine, vinorelbine, and zorubicin, or their respective derivatives or analogs thereof. Several of the above indicated drugs are now administered simultaneously for cancer therapy and, consequently, it is also envisioned that more than one cytostatic and/or cytotoxic drug can be comprised in compositions of the present invention.

As mentioned above, HIF inhibitors render cancer cells more vulnerable to chemotherapy and radiation therapy. Thus, to effectively treat a hyperproliferative disease or disorder, the compounds of the present invention can be co-administered with other active medicinal agents and/or administered in conjunction with other anticancer, antitumor, or antiproliferative disease therapies. In one aspect, the invention provides a method for treating a hyperproliferative disease or disorder comprising administering a compound according to the invention to a patient prior to, during and/or after said patient was subjected to a radiation therapy, a chemotherapy, an immunotherapy, a laser/microwave thermotherapy or a gene therapy using antisense DNA and RNA (for examples see Moeller et al., Cancer Cell 2004 5429-441).

In a further aspect the invention provides, as already outlined above, the use of a compound according to the invention or a composition according to the invention for the preparation of a medicament for the treatment or prevention of a disease or disorder selected from the group consisting of an inflammatory disease, a hyperproliferative disease or disorder, a hypoxia related pathology such as e.g. diabetic retinopathy, ischemic reperfusion injury, ischemic myocardial and limb disease, ischemic stroke, sepsis and septic shock (see, e.g. Liu F Q, et al., Exp Cell Res. 2008 Apr. 1; 314(6):1327-36); and a disease characterized by pathophysiological hyper-vascularisation, such as e.g. angiogenesis in osteosarcoma (see, e.g.: Yang, Qing-cheng et al., Dier Junyi Daxue Xuebao (2008), 29(5), 504-508), macular degeneration, in particular, age-related macular degeneration and vasoproliferative retinopathy (see e.g. Kim J H, et al., J Cell Mol. Med. 2008 Jan. 19).

As was already mentioned above, HIF inhibitors, such as the compounds of the invention, are useful to treat inflammatory disease or disorder. For example, it was shown that oxygen-dependent HIF isoforms are strongly upregulated in psoriatic skin (see e.g. Rosenberger C, et al., Invest Dermatol. 2007 October; 127(10):2445-52). Furthermore it was shown that a HIF inhibitor, neovastat, inhibits the airway inflammation in asthma (see e.g., Lee S Y, et al., Vascul Pharmacol. 2007 November-December; 47(5-6):313-8). Furthermore, recent evidence also shows that HIF participates under hypoxic conditions in joint inflammation and destruction in rheumatoid arthritis (see e.g., Alin, J. K., et al., Rheumatology (Oxford, United Kingdom) (2008), 47(6), 834-839). Thus, in a preferred embodiment of the use of the invention, the inflammatory disease is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichtyosises; alopecia greata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia greata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and periorale dermatitis. Therefore, a further preferred embodiment of the present invention encompasses a combination of one or more compounds of the present invention and medication in current use for treating such inflammatory diseases or conditions, which can be determined by a person skilled in the art of pharmacological sciences. Such therapeutics for combination can be selected e.g. from a group of anti-inflammatory steroids, antioxidants, therapeutic antibodies or fusion proteins that sequester or bind to certain cytokines or cellular epitopes associated with inflammatory processes, or a dihydrofolate reductase inhibitor like methotrexate.

The compounds of the invention show anti-proliferative effects. Furthermore, HIF inhibitors, such as the compounds of the invention are effective medicaments for the treatment of various cancer diseases (see review article by e.g. Gregg L. Semenza, Nature Reviews, October 2003, vol. 3 and also review article by N. J. Mabjeesh et al., Histol. Histopathol (2007), 22:559-572). Thus, also preferred is the use of the invention wherein the hyperproliferative disease is selected from the group consisting of a tumour or cancer disease, precancerosis, dysplasia, histiocytosis, a vascular proliferative disease and a virus-induced proliferative disease. Thus, in one preferred embodiment of the use of the invention the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T- cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal (in particular malignant renal cell carcinoma (RCC)), uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

The precancerosis treatable with the compounds of the present invention are preferably selected from the group consisting of precancerosis, in particular actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), leukoplakia, endometrial hyperplasia (grade III), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), leukoplakia; carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is can be found in e.g. the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Estrogen receptor refers to a group of receptors which are activated by the hormone 17β-estradiol (estrogen). Two types of estrogen receptor exist: ER which is a member of the nuclear hormone family of intracellular receptors and the estrogen G protein coupled receptor GPR30 (GPER), which is a G-protein coupled receptor. Estrogen and the estrogen receptors have been implicated in breast cancer, ovarian cancer, colon cancer, prostate cancer and endometrial cancer and other diseases. As the compounds of the invention are capable of inhibiting estrogen receptor-mediated transcriptional activity, they can be used to treat said diseases.

Thus, in a further preferred embodiment, the hyperproliferative disorders treatable according to the invention are those which benefit from a reduced estrogen receptor signalling, i.e. disorders associated with an increased estrogen receptor signaling, if compared to healthy tissue. This particular suitability of the compounds of the present invention is based on the fact, that the compounds of the present invention potentially through inhibiting cellular replication but possibly also through an additional activity of the compounds of the present invention exert an inhibition of estrogen receptor signaling. Thus, preferred diseases, conditions and/or disorders which can be treated are selected from the group consisting of mammary tumors, endometrial tumors and tumors of the uterus. Whether a disease is associated an increased estrogen receptor activity can be measured by a variety of art known methods including determination of ER expression level in the diseased tissue by, e.g. immunological methods, which determine the amount of expressed protein, by methods determining the amount of transcribed ER encoding nucleic acids, e.g. RT-PCR, Northern-blots, nuclear run-ons etc., and determining the activity of a nucleic acid construct comprising an ER-receptor recognition element, which drives expression of a detectable reporter, e.g. CAT, luciferase, GFP etc as described in more detail in the Experimental Section below. Preferably, the disorders which benefit from a reduced estrogen receptor signaling are those, which show in the diseased tisse an increase in estrogen receptor signaling by at least 10%, preferably by at least 20%, 30%, 40%, 50%, 60%, 70%, if compared to healthy tissue. Preferably this increase is measured on the basis of a nucleic acid comprising an ER-receptor recognition element and the increase of the expression of a reporter driven by this element.

In therapeutic use as an antagonist of estrogen receptor signaling, acting through inhibition of cellular replication, the compounds utilized in the use of the invention are administered at the initial dosage of about 0.02 mg/kg to about 20 mg/kg daily. A daily dose range of about 0.05 mg/kg to about 2 mg/kg is preferred, with a daily dose range of about 0.05 mg/kg to about 1 mg/kg being most preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

### Salts/Esters

The compounds within the compositions or compounds usable according to the present invention can be present as salts or esters, in particular pharmaceutically acceptable salts or esters. Pharmaceutically acceptable salts of the compounds of the invention include suitable acid addition or base salts thereof A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Isotopes

The compounds of the present invention may also contain unnatural proportions of atomic isotopes at one or more of the atoms that constitute such compounds. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ³H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

All isotopic variations of the compounds and compositions of the present invention, whether radioactive or not, are intended to be encompassed within the scope of the present invention.

### Solvates

The present invention also includes solvate forms of the compounds within the compositions or compounds according to any of general formulas (I) through (III) usable according to the present invention. The terms used in the claims encompass these forms.

### Polymorphs

The invention furthermore relates to compounds within the compositions of the present invention or compounds according to formula (I) usable according to the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

A compound according to the invention can be administered by various well known routes, including oral, rectal, intragastrical, intracranial and parenteral administration, e.g. intravenous, intramuscular, intranasal, intradermal, subcutaneous, and similar administration routes. Parenteral administration and particular intravenous administration, preferably by depot injection, is preferred. Depending on the route of administration different pharmaceutical formulations are required and some of those may require that protective coatings are applied to the drug formulation to prevent degradation of a compound of the invention in, for example, the digestive tract.

Thus, preferably, a compound of the invention is formulated as a syrup, an infusion or injection solution, a tablet, a capsule, a capslet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

Particular preferred pharmaceutical forms for the administration of a compound of the invention are forms suitable for injectionable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Preferred diluents of the present invention are water, physiological acceptable buffers, physiological acceptable buffer salt solutions or salt solutions. Preferred carriers are cocoa butter and vitebesole. Besides the preferred excipients mentioned already above, also the following excipients can be chosen, without limitation, to be used with the various pharmaceutical forms of a compound of the invention:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

Other suitable excipients can be found in the Handbook of Pharmaceutical Excipients, published by the American Pharmaceutical Association, which is herein incorporated by reference.

It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. It is contemplated that the average daily dosage of a compound of the invention in the therapeutic or prophylactic use of the invention should be in the range of about 0.1 mg to about 3 g. However, in a preferred use of the present invention a compound of the invention is administered to a subject in need thereof in an amount ranging from 1.0 to 1000 mg, preferably ranging from 10 to 500 mg preferably ranging from 50 to 200 mg. The duration of therapy and the dosing frequency with a compound of the invention will vary, depending on the severity of the disease being treated and the condition and idiosyncratic response of each individual patient.

As is known in the art, the pharmaceutically effective amount of a given composition will also depend on the administration route. In general the required amount will be higher, if the administration is through the gastrointestinal tract; e.g. by suppository, rectal, or by an intragastric probe, and lower if the route of administration is parenteral, e.g. intravenous. Typically, a compound of the invention will be administered in ranges of 50 mg to 3 g, preferably 50 mg to 500 mg, if rectal or intragastric administration is used and in ranges of 10 to 500 mg, if parenteral administration is used.

If a person is know to be at risk of developing a disorder treatable with a compound of the invention, a prophylactic administration of the pharmaceutical composition according to the invention may be possible. In these cases, the respective compound of the invention is preferably administered in above outlined preferred and particular preferred doses on a daily basis. This administration can be continued until the risk of developing the respective disorder has lessened. In most instances, however, a compound of the invention will be administered once a disease/disorder has been diagnosed. In these cases it is preferred that a first dose of a compound of the invention is administered one, two, three or four times daily. Preferably the administration is discontinued for one day, one week or one month and then repeated until the symptoms of the respective disease are no longer worsening or until they are improving.

Within the meaning of this invention, a combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week. This invention also envisions the quaternization of any basic nitrogen-containing groups of the compounds disclosed herein. Water or oil-soluble or dispersible products may be obtained by such quaternization.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples and figures are merely illustrative of the present invention and should not be construed to limit the scope of the invention as indicated by the appended claims in any way.

### BRIEF DESCRIPTION OF THE FIGURES

FIG. 1: Depicts the fluorescence-assisted cell sorting (FACS) data obtained in example 8 described below.

### EXPERIMENTAL SECTION

All starting materials used in the examples are either commercially available or can be synthesized by the average skilled person trained in organic chemistry without undue burden following routine laboratory practice as outlined, for example in example 1.

Besides the guidance provided in example 1, alternative synthetic transformations that may be employed in the synthesis of compounds of formulas I-III and in the synthesis of intermediates involved in the synthesis of compounds of formulas I-III are known by or accessible to one skilled in the art. Collections of synthetic transformations may be found in compilations, such as: J. March. Advanced Organic Chemistry, 4th ed.; John Wiley: New York (1992) R. C. Larock. Comprehensive Organic Transformations, 2nd ed.; Wiley-VCH: New York (1999); F. A. Carey; R. J. Sundberg. Advanced Organic Chemistry, 2nd ed.; Plenum Press: New York (1984) T. W. Greene; P. G. M. Wuts. Protective Groups in Organic Synthesis, 3rd ed.; John Wiley: New York (1999). L. S. Hegedus. Transition Metals in the Synthesis of Complex Organic Molecules, 2nd ed.; University Science Books: Mill Valley, Calif. (1994) L. A. Paquette, Ed. The Encyclopedia of Reagents for Organic Synthesis; John Wiley: New York (1994). A. R. Katritzky; 0. Meth-Cohn; C. W. Rees, Eds. Comprehensive Organic Functional Group Transformations; Pergamon Press: Oxford, UK (1995). G. Wilkinson; F. G A. Stone; E. W. Abel, Eds. Comprehensive Organometallic Chemistry; Pergamon Press: Oxford, UK (1982). B. M. Trost; I. Fleming. Comprehensive Organic Synthesis; Pergamon Press: Oxford, UK (1991) A. R. Katritzky; C. W. Rees Eds. Comprehensive Heterocylic Chemistry; Pergamon Press: Oxford, UK (1984) A. R. Katritzky; C. W. Rees; E. F. V. Scriven, Eds. Comprehensive Heterocylic Chemistry; Pergamon Press: Oxford, UK (1996). C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using SpotFire, and REACCS.

The compounds herein were named according to IUPAC standard using the software AutoNom Standard For ISIS/Draw Add-In.

### Example 1

### Synthesis of the Compounds of the Invention

The compounds according to general formulas (I) through (III) according to the present invention may be prepared for example according to the following scheme:

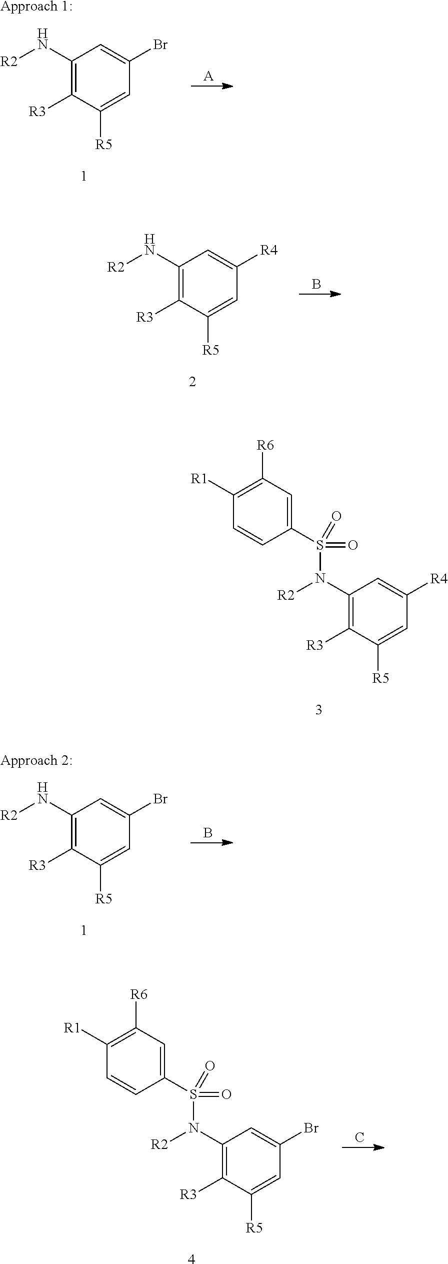

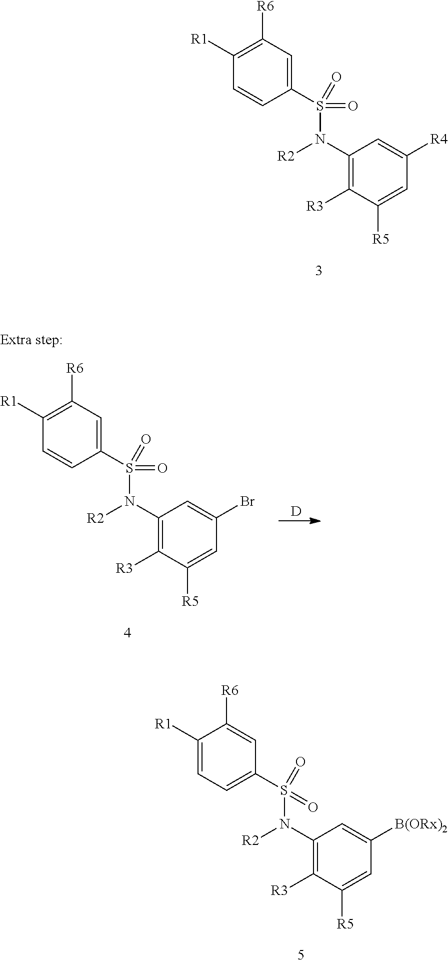

wherein R¹ through R⁶ are as defined in the claims, or have the particularly preferred meanings as defined herein and Rx is H and/or pinacolato.

The general reaction schemes indicated above are as follows:

General Procedure A. A flask charged with 1 (1.0 equiv), Pd(dppf)Cl2.CH₂Cl₂ (0.1 equiv), and the corresponding heteroaromatic or aromoatic boronic acid or boronic ester (1.2 equiv) in 1:1 (v/v) Toluene/MeOH was purged with N₂ under vigorous stirring, and 2.0 M aqueous K₂CO₃ solution (2.5 equiv) were added slowly. The mixture was heated at 90° C. for 4 h. After cooling, the mixture was purified by flash column chromatography (silica gel, EtOAc/heptane, 1:5 to 1:3) to give the derivatives 2 (43-68% yield).

General Procedure B. A flask charged with aniline derivatives 1 or 2 (1.0 equiv), the corresponding sulfonyl chloride (1.1 equiv) and pyridine (10 equiv) in CH₂Cl₂ was stirred at room temperature for 12 h. The mixture was extracted with concentrated aqueous NH₄Cl, washed with brine and dried over MgSO₄. The organic solvents were evaporated and the crude product was purified by preparative HPLC or by flash column chromatography (silica gel, EtOAc/heptane) to give the derivatives 3 or 4 (70% to quantitative yield).

General Procedure C. A 2-5 ml microwave reaction vessel charged with 4 (1.0 equiv), Pd(dppf)Cl₂.CH₂Cl₂ (0.1 equiv), and the corresponding heteroaromatic or aromatic boronic acid or boronic ester (1 equiv) in dry MeOH was purged with N₂ under vigorous stirring and 2.0 M aqueous K₂CO₃ solution (2.5 equiv) were added slowly. The vessel was sealed and the mixture was heated in a microwave oven for 30 min at 90° C. (CEM Discover Microwave system, set to Pₘₐₓ=150W). After cooling, the mixture was diluted with CH₂Cl₂, filtered through celite and eluted with CH₂Cl₂. The organic solvents were evaporated and the crude product was purified by preparative HPLC or by flash column chromatography (silica gel, EtOAc/heptane,) to give the derivatives 3 (42-90% yield).

General Procedure D. Approach 2 can optionally include an extra step as shown. Bispinacolato diboron (2.5 equiv), Pd(dppf)Cl₂.CH₂Cl₂ (0.1 equiv), and KOAc (3.0 equiv) were added to a flask containing compounds 4 (1.0 equiv) in degassed 1,4-dioxane. The reaction mixture was heated to 95° C. and stirred for 4 hours. Upon completion, the reaction mixture was diluted with EtOAc (50 ml), filtered through a short column of silica gel, and further eluted with EtOAc. The combined organic solvent was washed with H₂O and brine, dried over anhydrous MgSO₄ and concentrated in vacuum. The resulting residue was purified by flash column chromatography (silica gel, EtOAc/heptane, 1:3) to yield the boronate esters 5 (50-88% yield) as a cream or white solid. In a subsequent step, compounds 5 were transformed with a heteroaromatic or aromatic bromide to 3 following procedures A or C. Substituents R¹, R⁴ and/or R⁶, which may undergo unwanted reactions when carrying out a synthesis according to approach 1 or 2 may be protected by a conventional protecting group, which is not cleaved during the reactions according to approach 1 or 2 but is cleavable under known conditions. The skilled person is aware of a large variety of protection groups, which can be employed in organic synthesis. Protecting groups are reviewed in, for example, Wuts, P. G. M. and Greene, T. W., Protective Groups in Organic Chemistry, 3^{rd} Ed., 1999; Wily & Sons Inc. and in Kocienski, P. J., Protecting groups; 2″ Ed., 2000, Thieme Medical Publishing. Protecting groups are organized in these reference books according to the functionalities that are protected as well as according to the conditions which remove the respective protecting groups selectively. Particularly preferred protective groups, which can be used, are:
(i) protective group removed at acidic conditions, preferably at a pH between 4 and 6, which is selected from the group consisting of Boc or Trityl protecting groups;
(ii) a protecting group removed by a nucleophile, which is selected from the group consisting of Fmoc or Dde protecting groups;
(iii) a protecting group removed by hydrogenolysis consisting of the allyl type, the tert-butyl type, the benzyl type or Dmab (4-{N-[1-(4,4-dimethyl-2,6-dioxocyclohexylidene)-3-methylbutyl]amino}benzyl ester);
(iv) a protecting group removed by radiation, which is selected from the group consisting of nitroveratryloxy carbonyl, nitrobenzyloxy carbonyl, dimethyl dimethoxybenzyloxy carbonyl, 5-bromo-7-nitroindolinyl, o-hydroxy-α-methyl cinnamoyl, and 2-oxymethylene anthraquinone.

### Synthesis of Building Blocks

The toluidine building blocks (1) are commercially available, but can also be synthesized according to standard functionalization or transformation protocols, described in the literature cited above, and normally known to those ordinary skilled in the art of organic synthesis.

Numerous aromatic or heteroaromatic boronic acids or esters useful in Procedures A and C for palladium catalyzed coupling reactions are commercially available, or they can be synthesized according to standard functionalization or transformation protocols, described in the literature cited above, and normally known to those ordinary skilled in the art of organic synthesis. In particular, such boronic acid/ester intermediates can be generated e.g. from their corresponding aromatic or heteroaromatic halide precursors, as exemplified in Procedure D.

Numerous 5- and 6-membered heteroaromatic sulfonic acids and their corresponding activated derivatives, as used in Procedure B and there exemplified for sulfonylchlorides, are commercially available or they can be synthesized according to standard functionalization or transformation protocols, described in the literature cited above and normally known to those ordinary skilled in the art of organic synthesis. (Particular methods are e.g. described in: Caldwell, W T et al., J. Med. Chem. (1962), Vol. 6, p. 58 ff; Caldwell, W T et al., JACS (1959), Vol. 81, p. 5166 ff; Roblin, R O et al., JACS (1950), Vol. 72, p. 4890 ff; Hitoshi K et al., US Patent (1998), U.S. Pat. No. 5,811,571; WO 2006/090244, p. 38 ff; Janosik T et al., THL (2006), Vol. 62, p. 1699 ff; Allred GD, SERMACS (2007), Lanny Liebeskind Cope Scholar Award Symposium II, 222. Sulfonyl Fluorides; and references cited therein). If desired, it is also possible to generate sulfonamides 3 and/or 4 directly from the corresponding thiols (Wright S W et al., JOC (2006), Vol. 71, p. 1080 ff).

### Example 2

### HPLC/MS Analysis of the Compounds of the Invention

The compounds were analyzed as follows:

Measured via HPLC/MS, using a Waters X-bridge C₁₈-column, 5 μm particle size, 4.6×150 mm (diameter×length) at a flow rate of 1.75 ml/min with a linear gradient (water to acetonitrile, 0.2% formic acid as modifier) from initially 99:1 to 1:99 over 9.10 min, then hold for 1.80 min. Mass signals were determined using a Waters 3100 Mass Detector.

### Example 3

### General Cell Culture Maintenance and Cell Proliferation Assays

MCF-7 human breast adenocarcinoma cells and HL-60 acute promyelocytic leukaemia cells were obtained from ATCC (LGC Promochem). HG-1 multiple myeloma cells were obtained from Dr. D. Hose (DKFZ Heidelberg). The CellSensor® HRE-bla HCT116 cell line (colorectal carcinoma) was obtained from Invitrogen.

Cells were grown under humidified 95% air, 5% CO₂ at 37° C. in Dulbecco's modified Eagle's medium (DMEM: MCF-7) or RPMI 1640 medium (RPMI: HL-60, HG-1) supplemented with 10% fetal bovine serum (FBS), 100 U/ml penicillin and 100 μg/ml streptomycin, 2 mM L-glutamine, and 2 ng/ml IL-6 (for the HG-1 cell line only). The HCT116 cell line was grown under the same atmospheric conditions in McCoy's 5A medium supplemented with 10% FBS and penicillin/streptomycin as above, in addition to blasticidin as a selection marker at a final concentration of 5 μg/ml.

Cell proliferation experiments were carried out in 96-well tissue culture plates with seeding of 2000 cells/well (MCF-7) or 1000 cells/well (HL-60, HG-1) in 100 μl of the relevant medium. Cells were subsequently incubated under the conditions mentioned for 24 h prior to addition of compounds.

For the determination of compound EC₅₀ values, 10 μl compounds at 11× concentrations in 5.5% DMSO were added to the wells at various concentrations yielding a final constant percentage of 0.5% DMSO at the desired 1× compound concentration. As a positive control, cells were treated with 5.5% DMSO. The cells were then incubated for a further 72 h prior to the measurement.

To determine the degree of inhibition of cell proliferation, cells were treated with ATPlite solution according to the manufacturer's instructions (PerkinElmer, ATPlite 1-step Luminescence ATP Detection Assay System) and luciferase readout measured on an Envision HTS multilabel plate reader (PerkinElmer) in luminescence mode according to established protocols. Raw data were imported into an ActivityBase database (IDBS, ID Business Solutions) and EC₅₀ values calculated using the IDBS program ActivityBase XE. Table 2A and 2B show the IC₅₀ values obtained in the above-outlined cell proliferation assay for exemplary compounds of the invention. The cell proliferation assay data show that the compounds of the invention can inhibit cell proliferation in cancer cell lines.

TABLE 2A
MCF7 cells

| | | | | Mass |
|---|---|---|---|---|
| Example | | | Retention time (HPLC) | Spectromety |
| no. | IUPAC Name | EC₅₀ | [min] | MH+ |
| 1 | 4-Methyl-N-[2-methyl-5-(1H-tetrazol- | “+” | 5.87 | 330 |
| | 5-yl)-phenyl]-benzenesulfonamide | | | |
| 2 | 4-Methyl-N-(2-methyl-5-tetrazol-1-yl- | “+” | 6.27 | 330 |
| | phenyl)-benzenesulfonamide | | | |
| 3 | 4-Methyl-N-[2-methyl-5-(5-methyl-furan- | “+++” | 8.27 | 343 |
| | 2-yl)-phenyl]-benzenesulfonamide | | | |
| 4 | N-(5-Furan-2-yl-2-methyl-phenyl)-4- | “+++” | 7.92 | 328 |
| | methyl-benzenesulfonamide | | | |
| 5 | N-(5-Furan-3-yl-2-methyl-phenyl)-4- | “+++” | 7.73 | 328 |
| | methyl-benzenesulfonamide | | | |
| 6 | 4-Methyl-N-[2-methyl-5-(2-methyl-2H- | “+” | 6.88 | 344 |
| | tetrazol-5-yl)-phenyl]-benzenesulfonamide | | | |
| 7 | N-(4,3′-Dimethyl-biphenyl-3-yl)-4-methyl- | “++” | 8.7 | 352 |
| | benzenesulfonamide | | | |
| 8 | 4-Methyl-N-(2-methyl-5-thiophen-3-yl- | “+++” | 8.12 | 344 |
| | phenyl)-benzenesulfonamide | | | |
| 9 | N-[5-(6-Methoxy-pyridin-2-yl)-2-methyl- | “++” | 8.2 | 370 |
| | phenyl]-4-methyl-benzenesulfonamide | | | |
| 10 | N-(4,4′-Dimethyl-biphenyl-3-yl)-4-methyl- | “+” | 8.72 | 352 |
| | benzenesulfonamide | | | |
| 11 | 4-Methyl-N-[2-methyl-5-(5-methyl-thiophen- | “+++” | 8.65 | 358 |
| | 2-yl)-phenyl]-benzenesulfonamide | | | |
| 12 | N-[5-(2,4-Dimethyl-thiazol-5-yl)-2- | “+++” | 7.07 | 373 |
| | methyl-phenyl]-4-methyl- | | | |
| | benzenesulfonamide | | | |
| 13 | N-[5-(2,5-Dimethyl-thiophen-3-yl)-2- | “+” | 8.63 | 388 |
| | methyl-phenyl]-4-methoxy- | | | |
| | benzenesulfonamide | | | |
| 14 | 4-Methoxy-N-(2-methyl-5-thiophen-3- | “+++” | 7.72 | 360 |
| | yl-phenyl)-benzenesulfonamide | | | |
| 15 | 4-Methyl-N-(2-methyl-5-pyrrol-1-yl- | “+++” | 7.92 | 327 |
| | phenyl)-benzenesulfonamide | | | |
| 16 | N-[5-(3,5-Dimethyl-isoxazol-4-yl)-2- | “+++” | 7.03 | 373 |
| | methyl-phenyl]-4-methoxy- | | | |
| | benzenesulfonamide | | | |
| 17 | 4-Methoxy-N-[2-methyl-5-(2-methyl- | “+++” | 8.23 | 374 |
| | thiophen- | | | |
| | 3-yl)-phenyl]-benzenesulfonamide | | | |
| 18 | N-[5-(6-Methoxy-pyridin-3-yl)-2-methyl- | “+” | 7.73 | 368.94 |
| | phenyl]-4-methyl-benzenesulfonamide | | | |
| 19 | 4-Methyl-N-(2-methyl-5-pyridin-2-yl- | “++” | 5.94 | 338, 340 |
| | phenyl)-benzenesulfonamide | | | |
| 20 | 4-Methoxy-N-(2-methyl-5-oxazol-5-yl- | “+++” | 6.36 | 345 |
| | phenyl)-benzenesulfonamide | | | |
| 21 | 4-Methoxy-N-(2-methyl-5-pyrrol-1-yl- | “+++” | n.d. | n.d. |
| | phenyl)-benzenesulfonamide | | | |
| 22 | 4-Methyl-N-(2-methyl-5-[1,2,4]triazol- | “+” | 5.26 | 329 |
| | 4-yl-phenyl)-benzenesulfonamide | | | |
| 23 | 4-Methyl-N-[2-methyl-5-(4-methyl-thiophen- | “+++” | 8.46 | 358 |
| | 3-yl)-phenyl]-benzenesulfonamide | | | |
| 24 | 4-Methyl-N-[2-methyl-5-(3-methyl-thiophen- | “+++” | 8.53 | 358 |
| | 2-yl)-phenyl]-benzenesulfonamide | | | |
| 25 | 4-Methyl-N-[2-methyl-5-(2-methyl-furan- | “+++” | 8.11 | 342 |
| | 3-yl)-phenyl]-benzenesulfonamide | | | |
| 26 | N-[5-(3-Methoxy-pyridin-2-yl)-2-methyl- | “+” | 5.8 | 368.98 |
| | phenyl]-4-methyl-benzenesulfonamide | | | |
| 27 | 4-Methyl-N-[2-methyl-5-(3-trifluoromethyl- | “+” | 7.55 | 407 |
| | pyridin-2-yl)-phenyl]-benzenesulfonamide | | | |
| 28 | 4-Methyl-N-[2-methyl-5-(3-methyl-pyridin- | “+” | 4.95 | 353 |
| | 2-yl)-phenyl]-benzenesulfonamide | | | |
| 29 | N-[5-(2,5-Dimethyl-pyrrol-1-yl)-2-methyl- | “+” | 8.36 | 355 |
| | phenyl]-4-methyl-benzenesulfonamide | | | |
| 30 | 4-Methoxy-N-[2-methyl-5-(3-methyl- | “+++” | 7.14 | 358 |
| | pyrazol-1-yl)-phenyl]-benzenesulfonamide | | | |
| 31 | 4-Methyl-N-[2-methyl-5-(4-methyl-pyridin- | “+” | 5.28 | 352.98 |
| | 2-yl)-phenyl]-benzenesulfonamide | | | |
| 32 | 4-Methyl-N-[2-methyl-5-(5-methyl-pyridin- | “+” | 5.88 | 353 |
| | 2-yl)-phenyl]-benzenesulfonamide | | | |
| 33 | 4-Methyl-N-[2-methyl-5-(6-methyl-pyridin- | “++” | 5.4 | 353 |
| | 2-yl)-phenyl]-benzenesulfonamide | | | |
| 34 | N-[5-(5-Cyano-pyridin-3-yl)-2-methyl- | “+++” | 7 | 364 |
| | phenyl]-4-methyl-benzenesulfonamide | | | |
| 35 | N-[5-(5-Methoxy-pyridin-3-yl)-2-methyl- | “+” | 5.88 | 369 |
| | phenyl]-4-methyl-benzenesulfonamide | | | |
| 36 | N-[5-(6-Methoxy-4-methyl-pyridin-3- | “+” | 7.83 | 382.99 |
| | yl)-2-methyl-phenyl]-4-methyl- | | | |
| | benzenesulfonamide | | | |
| 37 | N-[5-(6-Ethoxy-pyridin-3-yl)-2-methyl- | “+” | 8.18 | 383 |
| | phenyl]-4-methyl-benzenesulfonamide | | | |
| 38 | 4-Methyl-N-[2-methyl-5-(4-trifluoromethyl- | “+” | 8.29 | 407 |
| | pyridin-2-yl)-phenyl]-benzenesulfonamide | | | |
| 39 | 4-Methyl-N-[2-methyl-5-(6-trifluoromethyl- | “++” | 8.36 | 407 |
| | pyridin-2-yl)-phenyl]-benzenesulfonamide | | | |
| 40 | N-[5-(2,4-Dimethyl-pyrrol-1-yl)-2-methyl- | “+” | n.d. | n.d. |
| | phenyl]-4-methoxy-benzenesulfonamide | | | |
| 41 | N-[5-(2-Cyano-pyrrol-1-yl)-2-methyl- | “++” | 7.95* | 368 |
| | phenyl]-4-methoxy-benzenesulfonamide | | | |
| 42 | N-[5-(6-Acetyl-pyridin-2-yl)-2-methyl- | “++” | 7.7 | 381 |
| | phenyl]-4-methyl-benzenesulfonamide | | | |
| 43 | N-[5-(5-Cyano-pyridin-2-yl)-2-methyl- | “+++” | 7.38 | 364 |
| | phenyl]-4-methyl-benzenesulfonamide | | | |
| 44 | N-[5-(5-Methoxy-pyridin-2-yl)-2-methyl- | “+++” | 6.91 | 369 |
| | phenyl]-4-methyl-benzenesulfonamide | | | |
| 45 | 4-Methyl-N-[2-methyl-5-(5-trifluoromethyl- | “+” | 8.39 | 407 |
| | pyridin-2-yl)-phenyl]-benzenesulfonamide | | | |
| 46 | N-[5-(5-Fluoro-6-methyl-pyridin-2-yl)- | “++” | 8.12 | 371 |
| | 2-methyl-phenyl]-4-methyl- | | | |
| | benzenesulfonamide | | | |
| 47 | N-[5-(5-Cyano-thiophen-2-yl)-2-methyl- | “+++” | 7.93 | 369 |
| | phenyl]-4-methyl-benzenesulfonamide | | | |
| 48 | N-[5-(3,5-Dimethyl-isoxazol-4-yl)-2- | “+++” | 7.34 | 357 |
| | methyl-phenyl]-4-methyl- | | | |
| | benzenesulfonamide | | | |
| 49 | N-[5-(5-Cyano-pyridin-2-yl)-2-methyl- | “+++” | 7.06 | 380 |
| | phenyl]-4-methoxy-benzenesulfonamide | | | |
| 50 | 4-Methoxy-N-[2-methyl-5-(2-methyl- | “+++” | 7.81 | 357 |
| | pyrrol-1-yl)-phenyl]-benzenesulfonamide | | | |
| 51 | N-[5-(5-Cyano-thiophen-2-yl)-2-methyl- | “+++” | 7.64 | 385 |
| | phenyl]-4-methoxy-benzenesulfonamide | | | |
| 52 | N-[5-(6-Cyano-pyridin-2-yl)-2-methyl- | “++” | 7.5 | 364 |
| | phenyl]-4-methyl-benzenesulfonamide | | | |
| 53 | N-[5-(5-Fluoro-pyridin-2-yl)-2-methyl- | “+++” | 7.63 | 356.75 |
| | phenyl]-4-methyl-benzenesulfonamide | | | |
| 54 | N-[5-(5-Acetyl-pyridin-2-yl)-2-methyl- | “+” | 7.07 | 381 |
| | phenyl]-4-methyl-benzenesulfonamide | | | |
| 55 | 4-Methyl-N-[2-methyl-5-(5-nitro-pyridin- | “+++” | 7.71 | 384 |
| | 2-yl)-phenyl]-benzenesulfonamide | | | |
| 56 | N-[5-(4-Cyano-thiophen-2-yl)-2-methyl- | “+++” | 7.45 | 385 |
| | phenyl]-4-methoxy-benzenesulfonamide | | | |
| 57 | N-(3′-Cyano-4-methyl-biphenyl-3-yl)- | “++” | 7.49 | 379 |
| | 4-methoxy-benzenesulfonamide | | | |
| 58 | N-(2′-Cyano-4-methyl-biphenyl-3-yl)- | “++” | 7.38 | 379 |
| | 4-methoxy-benzenesulfonamide | | | |
| 59 | N-[5-(3-Cyano-thiophen-2-yl)-2-methyl- | “++” | 7.67 | 369 |
| | phenyl]-4-methyl-benzenesulfonamide | | | |
| 60 | N-(4′-Cyano-4-methyl-biphenyl-3-yl)- | “+++” | 7.83 | 363 |
| | 4-methyl-benzenesulfonamide | | | |
| 61 | N-[5-(6-Cyano-pyridin-3-yl)-2-methyl- | “+++” | 7.02 | 380 |
| | phenyl]-4-methoxy-benzenesulfonamide | | | |
| 62 | N-[5-(2-Cyano-thiophen-3-yl)-2-methyl- | “+++” | 7.41 | 384 |
| | phenyl]-4-methoxy-benzenesulfonamide | | | |
| 63 | N-(4′-Cyano-4-methyl-biphenyl-3-yl)- | “+++” | 7.53 | 379 |
| | 4-methoxy-benzenesulfonamide | | | |
| 64 | N-[5-(5-Cyano-pyrimidin-2-yl)-2-methyl- | “++” | 7.27 | 365 |
| | phenyl]-4-methyl-benzenesulfonamide | | | |
| 65 | N-(3′,4′-Dicyano-4-methyl-biphenyl- | “+++” | 8.06 | 404 |
| | 3-yl)-4-methoxy-benzenesulfonamide | | | |
| 66 | N-[5-(6-Dimethylamino-pyridin-2-yl)- | “++” | 6.8 | 383 |
| | 2-methyl-phenyl]-4-methyl- | | | |
| | benzenesulfonamide | | | |
| 67 | N-[5-(4,6-Dimethyl-pyridin-2-yl)-2- | “+” | 5.14 | 367 |
| | methyl-phenyl]-4-methyl- | | | |
| | benzenesulfonamide | | | |
| 68 | N-[5-(5-Cyano-thiophen-3-yl)-2-methyl- | “+++” | 7.9 | 369 |
| | phenyl]-4-methyl-benzenesulfonamide | | | |
| 69 | N-[5-(4-Cyano-thiophen-3-yl)-2-methyl- | “+++” | 7.55 | 369 |
| | phenyl]-4-methyl-benzenesulfonamide | | | |
| 70 | N-[5-(3-Cyo-furan-2-yl)-2-methyl- | “+++” | 7.55 | 353 |
| | phenyl]-4-methyl-benzenesulfonamide | | | |
| 71 | N-(4′-Cyano-3′-fluoro-4-methyl-biphenyl- | “+++” | 8 | 381 |
| | 3-yl)-4-methyl-benzenesulfonamide | | | |
| 72 | N-(4′-Cyano-4,3′-dimethyl-biphenyl- | “+++” | 8.13 | 377 |
| | 3-yl)-4-methyl-benzenesulfonamide | | | |
| 73 | N-[5-(5-Cyano-thiophen-3-yl)-2-methyl- | “+++” | 7.53 | 385 |
| | phenyl]-4-methoxy-benzenesulfonamide | | | |
| 74 | N-[5-(5-Cyano-pyridin-3-yl)-2-methyl- | “+++” | 5.98 | 338.92 |
| | phenyl]-4-methoxy-benzenesulfonamide | | | |
| 75 | N-[5-(2-Cyano-pyridin-4-yl)-2-methyl- | “++” | 6.95 | 380 |
| | phenyl]-4-methoxy-benzenesulfonamide | | | |
| 76 | 4-Methyl-N-[2-methyl-5-(5-nitro-furan- | “+++” | 7.62 | 373 |
| | 2-yl)-phenyl]-benzenesulfonamide | | | |
| 77 | 4-Chloro-N-[5-(5-cyano-thiophen-2-yl)- | “+++” | 8.26 | 389 |
| | 2-methyl-phenyl]-benzenesulfonamide | | | |
| 78 | N-[5-(5-Cyano-thiophen-2-yl)-2-methyl- | “++” | 8.4 | 423 |
| | phenyl]-4-trifluoromethyl- | | | |
| | benzenesulfonamide | | | |
| 79 | N-[5-(5-Cyano-thiophen-2-yl)-2-methyl- | “+” | 8.48 | 439 |
| | phenyl]-4-trifluoromethoxy- | | | |
| | benzenesulfonamide | | | |
| 80 | N-[5-(4-Cyano-thiophen-2-yl)-2-methyl- | “+++” | 8.21 | 385 |
| | phenyl]-4-methoxy-benzenesulfonamide | | | |
| 81 | 4-Methoxy-N-(4′-methoxy-4-methyl- | “+++” | 7.92 | 384 |
| | biphenyl- | | | |
| | 3-yl)-benzenesulfonamide | | | |
| 82 | 4-Methoxy-N-(4-methyl-4′-methylsulfanyl- | “+” | 8.38 | 400 |
| | biphenyl-3-yl)-benzenesulfonamide | | | |
| 83 | 4-Methoxy-N-(4-methyl-4′-trifluoromethoxy- | “+” | 8.74 | 438 |
| | biphenyl-3-yl)-benzenesulfonamide | | | |
| 84 | N-(4′-Ethynyl-4-methyl-biphenyl-3-yl)- | “++” | 8.48 | 362 |
| | 4-methyl-benzenesulfonamide | | | |
| 85 | N-(4′-Cyano-4-methyl-biphenyl-3-yl)- | “+++” | 7.67 | 397 |
| | 3-fluoro-4-methoxy-benzenesulfonamide | | | |
| 86 | N-(4′-Cyano-4-methyl-biphenyl-3-yl)- | “+” | 7.67 | 392 (M − 1) |
| | 4-nitro-benzenesulfonamide | | | |
| 87 | 4-Amino-N-(4′-cyano-4-methyl-biphenyl- | “+++” | 6.85 | 364 |
| | 3-yl)-benzenesulfonamide | | | |
| 88 | N-(4′-Cyano-4-methyl-biphenyl-3-yl)- | “+++” | 8.04 | 381 |
| | 3-fluoro-4-methyl-benzenesulfonamide | | | |
| 89 | N-(4′-Cyano-3′-methoxy-4-methyl-biphenyl- | “+++” | 7.56 | 409 |
| | 3-yl)-4-methoxy-benzenesulfonamide | | | |
| 90 | N-(4′-Cyano-3′-hydroxy-4-methyl-biphenyl- | “+++” | 6.89 | 395 |
| | 3-yl)-4-methoxy-benzenesulfonamide | | | |
| 91 | N-[5-(5-Cyano-thiophen-2-yl)-2-methyl- | “++” | 7.78 | 373 |
| | phenyl]-4-fluoro-benzenesulfonamide | | | |
| 92 | N-[5-(5-Cyano-thiophen-2-yl)-2-methyl- | “+++” | 7.71 | 403 |
| | phenyl]-3-fluoro-4-methoxy- | | | |
| | benzenesulfonamide | | | |
| 93 | 4-Amino-N-[5-(5-cyano-thiophen-2-yl)- | “+++” | 6.86 | 370 |
| | 2-methyl-phenyl]-benzenesulfonamide | | | |
| 94 | 4-Methyl-N-[3-methyl-5-(5-methyl-thiophen- | “+++” | 8.56 | 358 |
| | 3-yl)-phenyl]-benzenesulfonamide | | | |
| 95 | N-(5-(5-cyanothiophen-3-yl)-2- | “+++” | 8.09 | 385 (M − 1) |
| | methylphenyl)-3-fluoro-4- | | | |
| | methylbenzenesulfonamide | | | |
| 96 | N-(5-(5-cyanothiophen-3-yl)-2- | “+” | 7.77 | 398 (M − 1) |
| | methylphenyl)-4-nitrobenzenesulfonamide | | | |
| 97 | 4-chloro-N-(4′-cyano-4-methylbiphenyl-3- | “+++” | 8.10 | 381 (M − 1) |
| | yl)benzenesulfonamide | | | |
| 98 | N-(5-(5-cyanothiophen-2-yl)-2- | “++” | 6.83 | 369 (M − 1) |
| | methylphenyl)-4- | | | |
| | hydroxybenzenesulfonamide | | | |
| 99 | N-(4′-cyano-3′-methoxy-4-methylbiphenyl-3- | “+++” | 7.99 | 409 (M − 1) |
| | yl)-3-fluoro-4-methylbenzenesulfonamide | | | |
| 100 | N-(4′-cyano-4-methylbiphenyl-3-yl)-4- | “+” | 6.38 | 363 (M − 1) |
| | hydroxybenzenesulfonamide | | | |
| 101 | N-(4′-cyano-3′-methoxy-4-methylbiphenyl-3- | “+” | 7.63 | 422 (M − 1) |
| | yl)-4-nitrobenzenesulfonamide | | | |
| 102 | 4-amino-N-(4′-cyano-3′-methoxy-4- | “+++” | 7.58 | 392 (M − 1) |
| | methylbiphenyl-3-yl)benzenesulfonamide | | | |
| 103 | N-(4′-cyano-3′-methoxy-4-methylbiphenyl-3- | “+++” | 8.46* | 425 (M − 1) |
| | yl)-3-fluoro-4-methoxybenzenesulfonamide | | | |
| 104 | N-(4′-cyano-4-methylbiphenyl-3-yl)-3- | “+” | 8.55* | 365 (M − 1) |
| | fluorobenzenesulfonamide | | | |
| 105 | N-(4′-cyano-4-methylbiphenyl-3-yl)-3- | “+” | 8.69* | 410 (M − 1) |
| | fluoro-4-nitrobenzenesulfonamide | | | |
| 106 | N-(5-(5-aminopyridin-2-yl)-2- | “+” | 6.24* | 352 (M − 1) |
| | methylphenyl)-4-methylbenzenesulfonamide | | | |
| 107 | N-(5-(2-methoxypyrimidin-4-yl)-2- | “+” | 8.43* | 368 (M − 1) |
| | methylphenyl)-4-methylbenzenesulfonamide | | | |
| 108 | N-(5-(5-cyanothiophen-2-yl)-2- | “++” | 8.15* | 415 |
| | methylphenyl)-3.4- | | | |
| | dimethoxybenzenesulfonamide | | | |
| 109 | N-(5-(5-cyanothiophen-2-yl)-2- | “+++” | 8.79* | 399 |
| | methylphenyl)-4-methoxy-3- | | | |
| | methylbenzenesulfonamide | | | |
| 110 | 4-amino-N-(4′-cyano-4-methylbiphenyl-3- | “+++” | 7.77* | 380 (M − 1) |
| | yl)-3-fluorobenzenesulfonamide | | | |
| 111 | 4-methyl-N-(2-methyl-5-(6-methyl-5- | “+++” | 8.96* | 398 |
| | nitropyridin-2-yl)phenyl)benzenesulfonamide | | | |
| 112 | N-(5-(5-cyanothiophen-2-yl)-2- | “+++” | 8.96* | 387 |
| | methylphenyl)-4-fluoro-3- | | | |
| | methylbenzenesulfonamide | | | |
| 113 | N-(5-(5-cyanothiophen-2-yl)-2- | “++” | 8.68* | 401 (M − 1) |
| | methylphenyl)-4-fluoro-3- | | | |
| | methoxybenzenesulfonamide | | | |
| 114 | N-(5-(5-cyanothiophen-2-yl)-2- | “+++” | 8.44* | 399 |
| | methylphenyl)benzo[d][1,3]dioxole-5- | | | |
| | sulfonamide | | | |
| 115 | N-(4′-cyano-4-methylbiphenyl-3-yl)-4- | “+++” | 8.60* | 392 |
| | (dimethylamino)benzenesulfonamide | | | |
| 116 | 4-cyano-N-(5-(5-cyanothiophen-2-yl)-2- | “++” | 8.36* | 378 (M − 1) |
| | methylphenyl)benzenesulfonamide | | | |

| | | | | |
|---|---|---|---|---|
| Reference: | EC₅₀ | <500 | nM: | +++ |
| | | 500-1000 | nM: | ++ |
| | | 1-10 | μM: | + |

Retention times marked with “*” were measured using an alternative method to the one given in Example
2. These retention times were determined as follows:
Measured via HPLC/MS, using a Waters X-bridge C₁₈-column, 5 μm particle size, 4.6 × 150 mm
(diameter × length) at a flow rate of 1.75 ml/min with a linear gradient (water to methanol, 0.2% formic
acid as modifier) from initially 99:1 to 1:99 over 9.10 min, then hold for 1.80 min. Mass signals were
determined using a Waters 3100 Mass Detector.

TABLE 2B
HG1 cells

| Example no. | EC₅₀ |
|---|---|
| 8 | +++ |
| 15 | +++ |
| 19 | +++ |
| 22 | +++ |
| 47 | +++ |
| 51 | +++ |
| 73 | +++ |

### Example 4

### Inhibition of Estrogen Receptor (ER) Signaling

To determine the effect of the compounds on estrogen receptor (ER)-mediated transcriptional activity a transfection assay using the MCF-7 cell line was performed. A luciferase-coupled ERE-tk-luc construct was obtained from Dr. G. Reid (EMBL).

In brief, MCF-7 cells were maintained as described previously and seeded on the first experimental day at a concentration of 3500 cells/well in 100 μl medium and incubated under standard conditions for 24 h. Following this initial incubation period, transfection was carried out using 5 ng of the ERE-tk-luc construct (per well) and Exgene 500 transfection reagent (Fermentas) in a final buffered solution containing 150 mM NaCl and 20 mM Tris pH 8.4. The plates were then maintained for 3-4 hours until cell culture conditions in the incubator before compounds were added at 11× concentrations to yield the final desired compound concentrations in 0.5% DMSO (in the same manner as for the proliferation assays described previously).

After an additional 24 h incubation period the degree of inhibition of ER-signaling was determined via a luciferase readout. Cells were treated with Britelite™ plus solution according to the manufacturer's instructions (PerkinElmer, britelite plus, Ultra-High Sensitivity Luminescence Reporter Gene Assay System) and luciferase levels measured on an Envision HTS multilabel plate reader (PerkinElmer) in luminescence mode according to established protocols. Raw data were imported into an ActivityBase database (IDBS, ID Business Solutions) and IC₅₀ values calculated using the IDBS program ActivityBase XE.

The results are depicted in Table 3. As shown in these experiments, the compounds of the invention are capable of inhibiting estrogen receptor element-mediated transcriptional activity in an ERα-positive cell line. The utility of ER-modulating agents for the treatment of breast, uterine or prostate cancer, as well as metastatic bone disease, is known from the literature (see for example, Park & Jordan (2002) Trends Mol. Med. 8(2): 82-88; Steiner et al. (2001) Urology 57(4 Suppl 1): 68-72 and Campisi et al. (1993) Eur. J. Gynaecol. Oncol. 14(6): 479-483.)).

TABLE 3

| Example no. | IC₅₀ |
|---|---|
| 4 | +++ |
| 5 | +++ |
| 15 | +++ |
| 43 | +++ |
| 47 | +++ |
| 49 | +++ |
| 60 | +++ |
| 72 | +++ |

| | | | | |
|---|---|---|---|---|
| Reference: | IC₅₀ | <500 | nM: | +++ |
| | | 500-1000 | nM: | ++ |
| | | 1-10 | μM: | + |

### Example 5

### Inhibition of In Vitro Tubulin Polymerization

To assess the degree of inhibition of in vitro tubulin polymerization, a tubulin solution (prepared in-house from porcine brain using a standard procedure involving cycles of polymerization/de-polymerization, see Castoldi & Popov (2003) Protein Expr. Purif. 32(1): 83-88) was incubated with varying concentrations of compounds under polymerizing conditions and kinetic experiments performed to determine the amount of polymerization (as measured by changes in OD at 340 nm) taking place over a 90 min period.

Compound solutions were diluted in double-distilled water to yield final 10× solutions containing 5% DMSO. 5 μl solutions were then added to the wells on a 384-well clear-bottom plate (Corning #3711, Corning Inc.) in preparation for the addition of tubulin. The tubulin solution (19.6 mg/ml, in 80 mM K-PIPES [pH 6.8], 1 mM MgCl₂, 1 mM EGTA) was re-suspended to a final concentration of 4 mg/ml with ice cold G-PEM buffer (80 mM PIPES [pH 6.8], 2 mM MgCl₂, 0.5 mM EGTA, 10% glycerol, 1 mM GTP) and maintained on ice for at least 1 min. 50 μl was then added to the pre-dispensed compound solutions, the plate orbital shaken on a medium setting for 5 s, and the first measurement begun immediately.

Experiments were carried out on a Safire²™ monochromator (Tecan) preset to a stable temperature of 37° C.; the degree of polymerization was determined by measuring the absorbance of the solutions at 340 nm every minute over the 90 min cycle. Reported inhibition values were based on the final absorbance at 90 min and calculated with reference to a vehicle control (0.5% DMSO) using the minimum and maximum signals obtained for this sample.

As demonstrated by these experiments (see Table 4), changes in the structural properties of the various examples can lead to a selection of compounds that are active in inhibiting cell proliferation and affect HIF-signaling, but that do, or do not, have tubulin inhibition properties, depending on whether this is desired in the target product profile.

TABLE 4

| Example no. | Inhibition (%) |
|---|---|
| 3 | + |
| 11 | +/− |
| 16 | +/− |
| 23 | + |
| 56 | + |
| 60 | − |
| 63 | − |
| 72 | − |

| | | | |
|---|---|---|---|
| Reference: | Inhibition at 10 μM | >50%: | + |
| | | 20-50%: | +/− |
| | | <20% | − |

### Example 6

### Inhibition of Activation of HIF Mediated Transcription under Hypoxic Conditions

Inhibition of an activated HIF signaling response under chemically-induced hypoxic conditions due to compound treatment was determined using the CellSensor® HRE-bla HCT-116 stably transfected reporter cell line from Invitrogen according to the manufacturer's instructions. HIF is a transcriptional factor composed of the constitutively expressed HIF1β subunit and one of three HIFα subunits (HIF1α, HIF2α, HIF3α). This assay is responsive towards HIFa activity in general. In one example, HIF-1, when stabilized by hypoxic conditions, upregulates several genes to promote cell survival in low-oxygen conditions. These include glycolysis enzymes, which allow ATP synthesis in an oxygen-independent manner, and vascular endothelial growth factor (VEGF), which promotes angiogenesis. HIF-1 acts by binding to HIF-responsive elements (HREs) in promoters that generally contain the sequence NCGTG.

Cells were maintained as described previously and seeded into 384-well, clear-bottom plates (Corning 3712) at 15000 cells/well in 32 μl assay medium (Opti-MEM [Invitrogen], 0.5% FBS, 100 U/ml penicillin, 100 μg/ml streptomycin, 0.1 mM non-essential amino acids [NEAA], 1 mM sodium pyruvate, 5 mM HEPES [pH 7.3]). Following a 2 h incubation period, compounds (4 μl) were subsequently added to the cells at 10× concentrations in 5% DMSO and incubated under normal conditions for 30 min. To induce hypoxic conditions, 4 μl of a 2 mM deferoxamine (DFO) solution was added to the cells followed by 24 h incubation under standard assay conditions (as described). Control wells included wells containing only medium (no cells) and wells treated with 0.5% DMSO instead of compound.

Prior to the readout, the Substrate Loading Solution was prepared as described in the manufacturer's protocol and 10 μl added to each well. Following a further 2 h incubation period at room temperature and in the dark, fluorescence was measured at two wavelengths (blue channel: ex. 409 nm, em. 460 nm; green channel: ex. 409 nm, em. 530 nm) on a PerkinElmer Envision HTS. For the analysis, the average signal of the cell-free wells at 460 nm and 530 nm was first subtracted from the blue and green channel data, respectively. The blue/green emission ratios were then calculated for each well, dividing the background-corrected blue emission values by the background-corrected green emission values. IC₅₀ values were determined from these ratios using GraphPad Prism (Prism 5, GraphPad software, Inc.).

The results of these experiments (see Table 5) show that the compounds of the invention are capable of inhibiting hypoxia regulated element-mediated transcriptional activity under hypoxic conditions. The compounds of this invention have a potency level that exceeds HIF-inhibitor compounds described in the prior art such as, e.g. the ProlX compound PX-478, the only small molecule HIF-inhibitor currently reported to undergo clinical trials.

TABLE 5

| Example no. | EC₅₀ |
|---|---|
| 3 | +++ |
| 8 | +++ |
| 23 | +++ |
| 43 | +++ |
| 51 | +++ |
| 68 | +++ |
| 71 | +++ |
| 73 | +++ |
| 95 | +++ |
| 103 | +++ |
| 112 | +++ |
| 114 | +++ |

| | | | | |
|---|---|---|---|---|
| Reference: | EC₅₀ | <500 | nM: | +++ |
| | | 500-1000 | nM: | ++ |
| | | 1-10 | μM: | + |

### Example 7

### Activation of Apoptosis

Activation of caspase-3/7 signal following treatment with test compounds was carried out using a Caspase-Glo® 3/7 kit (Promega). In brief, HG-1 cells were seeded at a concentration of 1000 cells/well in 50 μl medium into 96-well plates and incubated for 24 h under the conditions described previously. Compounds at various concentrations were then added to the wells (at all final concentrations 0.5% DMSO) and incubated for a further 24 h period. The plates were subsequently removed from the incubator and allowed to equilibrate to room temperature, after which 50 μl Caspase-Glo reagent was added to all wells, the plate shaken at 300 rpm for 30 s, and luminescence measured after a 30 min incubation period on an Envision HTS (PerkinElmer).

Following treatment with compounds of the invention, a clear activation of the caspase-3/7 response was observed (see Table 6), indicating that apoptosis is one factor in the observed decrease in cell proliferation due to the compound mechanisms of action.

TABLE 6

| Example no. | EC₅₀ |
|---|---|
| 47 | +++ |
| 51 | +++ |
| 56 | +++ |
| 60 | +++ |
| 63 | +++ |

| | | | | |
|---|---|---|---|---|
| Reference: | EC₅₀ | <500 | nM: | +++ |
| | | 500-1000 | nM: | ++ |
| | | 1-10 | μM: | + |

### Example 8

### Cell Cycle Analysis

To determine the effect on the cell cycle of HL-60 cells following treatment with compounds, fluorescence-assisted cell sorting (FACS) was used, sorting cells by nuclear staining.

A standard protocol using propidium iodide was utilized. In brief, cells were seeded into 6-well tissue culture plates at 2×10⁵ cells in 2 ml medium and incubated for 24 h at 37° C. under standard tissue culture conditions (humidified 95% air, 5% CO₂). Following this initial incubation period, compound solutions were added from stock solutions yielding final concentrations of 20 μM in 0.1% DMSO and the cells incubated after gentle mixing for further 24 h.

Cells were subsequently harvested, washed, and re-suspended in phosphate-bufferd saline (PBS, pH 7.4). Fixation was performed in ice-cold 70% ethanol while vortexing at half-speed and the solutions stored overnight at 4° C. The cells were then spun down, washed 3 times in PBS, and stained with a propidium iodide staining solution (20 μg/ml propidium iodide, 200 μg/ml RNase A, 0.1% Triton X-100 in PBS, pH 7.4). Finally, samples were passed through a 70 μm filter (BD Falcon) prior to measurement on a FACScan™ flow cytometer (Beckton Dickinson).

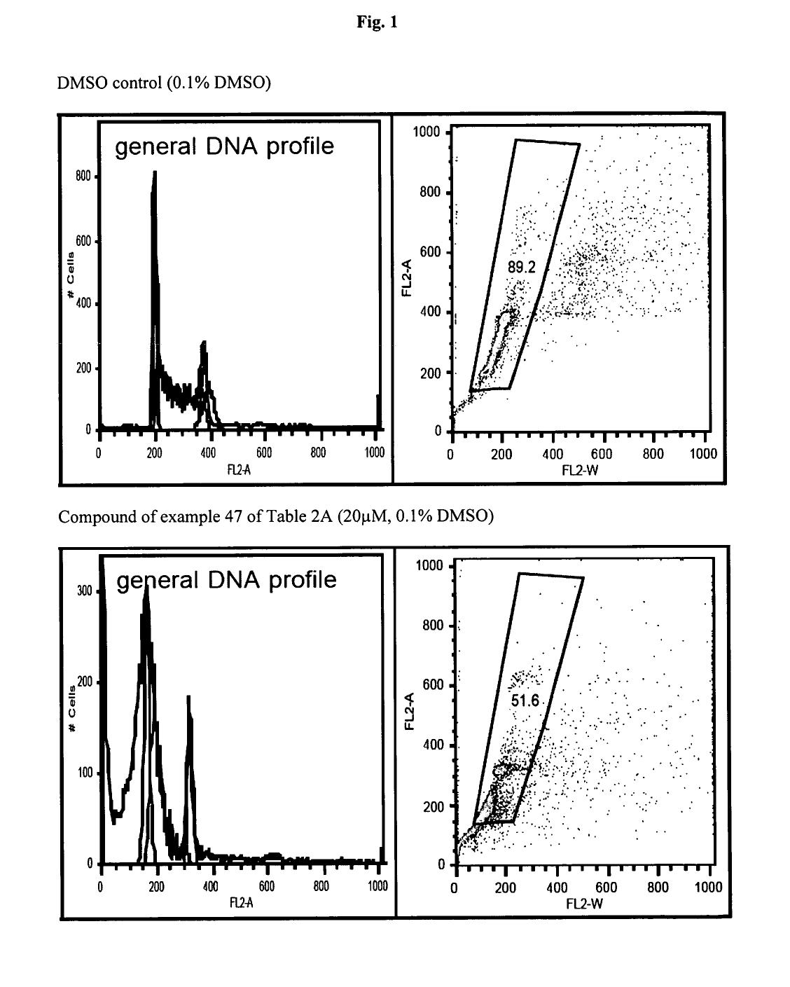

Together with the data showing caspase-3/7 activation, the FACS analysis shown for a representative compound demonstrates the appearance of a sub-2N population indicative of a cell cycle effect leading to apoptosis (see results depicted in FIG. 1). The ability of the compounds of the invention to induce apoptosis is a useful feature which can be exploited in chemotherapeutical applications.

### Example 9

### Further Useful Compounds According to the Invention

Further compounds of the invention are exemplified in Table 7, which are of particular use within the scope of the present invention. These compounds can be produced as outlined under example 1 and also according to manufacturing practice well known in organic chemistry as described above. These compounds will have a similar inhibitory activity as shown for the compounds listed in table 2A and tested in examples 3 to 8.

TABLE 7

| Example Number | IUPAC Name |
|---|---|
| 117 | 4-Methyl-N-[3-methyl-5-(5-methyl-furan-2-yl)-phenyl]-benzenesulfonamide |
| 118 | N-(3-Furan-2-yl-5-methyl-phenyl)-4-methyl-benzenesulfonamide |
| 119 | N-(3-Furan-3-yl-5-methyl-phenyl)-4-methyl-benzenesulfonamide |
| 120 | 4-Methyl-N-(3-methyl-5-thiophen-3-yl-phenyl)-benzenesulfonamide |
| 121 | 4-Methyl-N-[3-methyl-5-(5-methyl-thiophen-2-yl)-phenyl]-benzenesulfonamide |
| 122 | 4-Methyl-N-(3-methyl-5-thiophen-2-yl-phenyl)-benzenesulfonamide |
| 123 | N-[3-(2,4-Dimethyl-thiazol-5-yl)-5-methyl-phenyl]-4-methyl-benzenesulfonamide |
| 124 | 4-Methyl-N-(3-methyl-5-pyrrol-1-yl-phenyl)-benzenesulfonamide |
| 125 | N-[3-(3,5-Dimethyl-isoxazol-4-yl)-5-methyl-phenyl]-4-methyl-benzenesulfonamide |
| 126 | 4-Methyl-N-[3-methyl-5-(2-methyl-thiophen-3-yl)-phenyl]-benzenesulfonamide |
| 127 | 4-Methyl-N-(3-methyl-5-oxazol-5-yl-phenyl)-benzenesulfonamide |
| 128 | 4-Methyl-N-[3-methyl-5-(3-methyl-pyrazol-1-yl)-phenyl]-benzenesulfonamide |
| 129 | N-[3-(6-Cyano-pyridin-3-yl)-5-methyl-phenyl]-4-methyl-benzenesulfonamide |
| 130 | N-[3-(5-Cyano-pyridin-2-yl)-5-methyl-phenyl]-4-methyl-benzenesulfonamide |
| 131 | N-[3-(5-Methoxy-pyridin-2-yl)-5-methyl-phenyl]-4-methyl-benzenesulfonamide |
| 132 | N-[3-(5-Cyano-thiophen-2-yl)-5-methyl-phenyl]-4-methyl-benzenesulfonamide |
| 133 | N-[3-(5-Fluoro-pyridin-2-yl)-5-methyl-phenyl]-4-methyl-benzenesulfonamide |
| 134 | N-(4′-Cyano-5-methyl-biphenyl-3-yl)-4-methyl-benzenesulfonamide |
| 135 | N-(3′,4′-Dicyano-5-methyl-biphenyl-3-yl)-4-methyl-benzenesulfonamide |
| 136 | N-(4′-Cyano-3′-fluoro-5-methyl-biphenyl-3-yl)-4-methyl-benzenesulfonamide |
| 137 | N-(4′-Cyano-5,3′-dimethyl-biphenyl-3-yl)-4-methyl-benzenesulfonamide |
| 138 | N-[3-(2-Cyano-pyridin-4-yl)-5-methyl-phenyl]-4-methyl-benzenesulfonamide |
| 139 | 4-Methyl-N-[3-methyl-5-(5-nitro-furan-2-yl)-phenyl]-benzenesulfonamide |
| 140 | 4-Chloro-N-[3-(5-cyano-thiophen-2-yl)-5-methyl-phenyl]-benzenesulfonamide |
| 141 | N-(4′-Methoxy-5-methyl-biphenyl-3-yl)-4-methyl-benzenesulfonamide |
| 142 | N-(4′-Cyano-3′-hydroxy-5-methyl-biphenyl-3-yl)-4-methyl-benzenesulfonamide |
| 143 | N-(4′-Cyano-3′-methoxy-5-methyl-biphenyl-3-yl)-4-methyl-benzenesulfonamide |
| 144 | N-(4′-Cyano-3′-hydroxy-5-methyl-biphenyl-3-yl)-4-methoxy-benzenesulfonamide |
| 145 | N-(4′-Cyano-3′-methoxy-5-methyl-biphenyl-3-yl)-4-methoxy-benzenesulfonamide |
| 146 | N-[3-(5-Cyano-pyridin-2-yl)-5-methyl-phenyl]-4-methoxy-benzenesulfonamide |
| 147 | 4-Methoxy-N-[3-(5-methoxy-pyridin-2-yl)-5-methyl-phenyl]-benzenesulfonamide |
| 148 | N-[3-(5-Cyano-thiophen-2-yl)-5-methyl-phenyl]-4-methoxy-benzenesulfonamide |
| 149 | N-[3-(5-Fluoro-pyridin-2-yl)-5-methyl-phenyl]-4-methoxy-benzenesulfonamide |
| 150 | N-(4′-Cyano-5-methyl-biphenyl-3-yl)-4-methoxy-benzenesulfonamide |
| 151 | N-(3′,4′-Dicyano-5-methyl-biphenyl-3-yl)-4-methoxy-benzenesulfonamide |
| 152 | N-(4′-Cyano-3′-fluoro-5-methyl-biphenyl-3-yl)-4-methoxy-benzenesulfonamide |
| 153 | N-(4′-Cyano-5,3′-dimethyl-biphenyl-3-yl)-4-methoxy-benzenesulfonamide |
| 154 | N-[3-(2-Cyano-pyridin-4-yl)-5-methyl-phenyl]-4-methoxy-benzenesulfonamide |
| 155 | 4-Methoxy-N-[3-methyl-5-(5-nitro-furan-2-yl)-phenyl]-benzenesulfonamide |
| 156 | 4-Methoxy-N-(4′-methoxy-5-methyl-biphenyl-3-yl)-benzenesulfonamide |
| 157 | 4-Methoxy-N-(3-methyl-5-pyrrol-1-yl-phenyl)-benzenesulfonamide |
| 158 | N-[3-(3,5-Dimethyl-isoxazol-4-yl)-5-methyl-phenyl]-4-methoxy-benzenesulfonamide |
| 159 | 4-Methoxy-N-[3-methyl-5-(2-methyl-thiophen-3-yl)-phenyl]-benzenesulfonamide |
| 160 | 4-Methoxy-N-(3-methyl-5-oxazol-5-yl-phenyl)-benzenesulfonamide |
| 161 | 4-Methoxy-N-[3-methyl-5-(3-methyl-pyrazol-1-yl)-phenyl]-benzenesulfonamide |
| 162 | N-[3-(6-Cyano-pyridin-3-yl)-5-methyl-phenyl]-4-methoxy-benzenesulfonamide |
| 163 | 4-Methoxy-N-[3-methyl-5-(5-methyl-furan-2-yl)-phenyl]-benzenesulfonamide |
| 164 | N-(3-Furan-2-yl-5-methyl-phenyl)-4-methoxy-benzenesulfonamide |
| 165 | N-(3-Furan-3-yl-5-methyl-phenyl)-4-methoxy-benzenesulfonamide |
| 166 | 4-Methoxy-N-(3-methyl-5-thiophen-3-yl-phenyl)-benzenesulfonamide |
| 167 | 4-Methoxy-N-[3-methyl-5-(5-methyl-thiophen-2-yl)-phenyl]-benzenesulfonamide |
| 168 | 4-Methoxy-N-(3-methyl-5-thiophen-2-yl-phenyl)-benzenesulfonamide |
| 169 | N-[3-(2,4-Dimethyl-thiazol-5-yl)-5-methyl-phenyl]-4-methoxy-benzenesulfonamide |
| 170 | N-[3-(2,4-Dimethyl-thiazol-5-yl)-4-methyl-phenyl]-4-methoxy-N-methyl- |
| | benzenesulfonamide |
| 171 | 4-Methoxy-N-methyl-N-(4-methyl-3-thiophen-2-yl-phenyl)-benzenesulfonamide |
| 172 | 4,N-Dimethyl-N-[4-methyl-3-(5-methyl-furan-2-yl)-phenyl]-benzenesulfonamide |
| 173 | 4,N-Dimethyl-N-(4-methyl-3-thiophen-3-yl-phenyl)-benzenesulfonamide |
| 174 | 4-Methoxy-N-methyl-N-[4-methyl-3-(5-methyl-thiophen-2-yl)-phenyl]- |
| | benzenesulfonamide |
| 175 | 4-Methoxy-N-methyl-N-(4-methyl-3-thiophen-3-yl-phenyl)-benzenesulfonamide |
| 176 | N-(3-Furan-3-yl-4-methyl-phenyl)-4-methoxy-N-methyl-benzenesulfonamide |
| 177 | N-[3-(6-Cyano-pyridin-3-yl)-4-methyl-phenyl]-4-methoxy-N-methyl- |
| | benzenesulfonamide |
| 178 | 4-Methoxy-N-methyl-N-(4-methyl-3-oxazol-5-yl-phenyl)-benzenesulfonamide |
| 179 | 4-Methoxy-N-methyl-N-[4-methyl-3-(3-methyl-pyrazol-1-yl)-phenyl]- |
| | benzenesulfonamide |
| 180 | 4-Methoxy-N-methyl-N-[4-methyl-3-(5-methyl-furan-2-yl)-phenyl]- |
| | benzenesulfonamide |
| 181 | N-(3-Furan-2-yl-4-methyl-phenyl)-4-methoxy-N-methyl-benzenesulfonamide |
| 182 | 4-Methoxy-N-methyl-N-[4-methyl-3-(5-nitro-furan-2-yl)-phenyl]- |
| | benzenesulfonamide |
| 183 | 4-Methoxy-N-methyl-N-[4-methyl-3-(2-methyl-thiophen-3-yl)-phenyl]- |
| | benzenesulfonamide |
| 184 | N-[3-(3,5-Dimethyl-isoxazol-4-yl)-4-methyl-phenyl]-4-methoxy-N-methyl- |
| | benzenesulfonamide |
| 185 | N-[3-(2-Cyano-pyridin-4-yl)-4-methyl-phenyl]-4-methoxy-N-methyl- |
| | benzenesulfonamide |
| 186 | 4-Methoxy-N-(4′-methoxy-6-methyl-biphenyl-3-yl)-N-methyl-benzenesulfonamide |
| 187 | 4-Methoxy-N-methyl-N-(4-methyl-3-pyrrol-1-yl-phenyl)-benzenesulfonamide |
| 188 | N-(4′-Cyano-6,3′-dimethyl-biphenyl-3-yl)-4-methoxy-N-methyl-benzenesulfonamide |
| 189 | N-(4′-Cyano-6-methyl-biphenyl-3-yl)-4-methoxy-N-methyl-benzenesulfonamide |
| 190 | N-(3′,4′-Dicyano-6-methyl-biphenyl-3-yl)-4-methoxy-N-methyl-benzenesulfonamide |
| 191 | N-(4′-Cyano-3′-fluoro-6-methyl-biphenyl-3-yl)-4-methoxy-N-methyl- |
| | benzenesulfonamide |
| 192 | 4,N-Dimethyl-N-(4-methyl-3-thiophen-2-yl-phenyl)-benzenesulfonamide |
| 193 | 4-Methoxy-N-[3-(5-methoxy-pyridin-2-yl)-4-methyl-phenyl]-N-methyl- |
| | benzenesulfonamide |
| 194 | N-[3-(5-Cyano-thiophen-2-yl)-4-methyl-phenyl]-4-methoxy-N-methyl- |
| | benzenesulfonamide |
| 195 | N-[3-(5-Fluoro-pyridin-2-yl)-4-methyl-phenyl]-4,N-dimethyl-benzenesulfonamide |
| 196 | N-(4′-Cyano-3′-hydroxy-6-methyl-biphenyl-3-yl)-4-methoxy-N-methyl- |
| | benzenesulfonamide |
| 197 | N-[3-(5-Cyano-pyridin-2-yl)-4-methyl-phenyl]-4-methoxy-N-methyl- |
| | benzenesulfonamide |
| 198 | N-(4′-Cyano-3′-methoxy-6-methyl-biphenyl-3-yl)-4-methoxy-N-methyl- |
| | benzenesulfonamide |
| 199 | N-(4′-Cyano-3′-methoxy-6-methyl-biphenyl-3-yl)-4,N-dimethyl-benzenesulfonamide |
| 200 | N-(3-Furan-3-yl-4-methyl-phenyl)-4,N-dimethyl-benzenesulfonamide |
| 201 | 4,N-Dimethyl-N-[4-methyl-3-(3-methyl-pyrazol-1-yl)-phenyl]-benzenesulfonamide |
| 202 | N-[3-(3,5-Dimethyl-isoxazol-4-yl)-4-methyl-phenyl]-4,N-dimethyl- |
| | benzenesulfonamide |
| 203 | N-[3-(2,4-Dimethyl-thiazol-5-yl)-4-methyl-phenyl]-4,N-dimethyl- |
| | benzenesulfonamide |
| 204 | N-[3-(5-Methoxy-pyridin-2-yl)-4-methyl-phenyl]-4,N-dimethyl-benzenesulfonamide |
| 205 | 4,N-Dimethyl-N-(4-methyl-3-pyrrol-1-yl-phenyl)-benzenesulfonamide |
| 206 | 4,N-Dimethyl-N-(4-methyl-3-oxazol-5-yl-phenyl)-benzenesulfonamide |
| 207 | 4,N-Dimethyl-N-[4-methyl-3-(5-methyl-thiophen-2-yl)-phenyl]-benzenesulfonamide |
| 208 | N-[3-(5-Cyano-pyridin-2-yl)-4-methyl-phenyl]-4,N-dimethyl-benzenesulfonamide |
| 209 | 4,N-Dimethyl-N-[4-methyl-3-(2-methyl-thiophen-3-yl)-phenyl]-benzenesulfonamide |
| 210 | N-[3-(5-Cyano-thiophen-2-yl)-4-methyl-phenyl]-4,N-dimethyl-benzenesulfonamide |
| 211 | N-(3-Furan-2-yl-4-methyl-phenyl)-4,N-dimethyl-benzenesulfonamide |
| 212 | N-[3-(6-Cyano-pyridin-3-yl)-4-methyl-phenyl]-4,N-dimethyl-benzenesulfonamide |
| 213 | N-(4′-Cyano-6-methyl-biphenyl-3-yl)-4,N-dimethyl-benzenesulfonamide |
| 214 | N-(3′,4′-Dicyano-6-methyl-biphenyl-3-yl)-4,N-dimethyl-benzenesulfonamide |
| 215 | N-(4′-Cyano-3′-hydroxy-6-methyl-biphenyl-3-yl)-4,N-dimethyl-benzenesulfonamide |
| 216 | N-[3-(2-Cyano-pyridin-4-yl)-4-methyl-phenyl]-4,N-dimethyl-benzenesulfonamide |
| 217 | N-(4′-Cyano-3′-fluoro-6-methyl-biphenyl-3-yl)-4,N-dimethyl-benzenesulfonamide |
| 218 | N-(4′-Cyano-6,3′-dimethyl-biphenyl-3-yl)-4,N-dimethyl-benzenesulfonamide |
| 219 | 4,N-Dimethyl-N-[4-methyl-3-(5-nitro-furan-2-yl)-phenyl]-benzenesulfonamide |
| 220 | N-(4′-Methoxy-6-methyl-biphenyl-3-yl)-4,N-dimethyl-benzenesulfonamide |
| 221 | 4-Chloro-N-[3-(5-cyano-thiophen-2-yl)-4-methyl-phenyl]-N-methyl- |
| | benzenesulfonamide |
| 222 | 4-Methyl-N-[2-methyl-5-(1-methyl-1H-pyrrol-2-yl)-phenyl]-benzenesulfonamide |
| 223 | 4-Methoxy-N-[2-methyl-5-(1-methyl-1H-pyrrol-2-yl)-phenyl]-benzenesulfonamide |
| 224 | 4-Methyl-N-[2-methyl-5-(1-methyl-1H-pyrrol-3-yl)-phenyl]-benzenesulfonamide |
| 225 | 4-Methoxy-N-[2-methyl-5-(1-methyl-1H-pyrrol-3-yl)-phenyl]-benzenesulfonamide |
| 226 | 4-Methyl-N-[3-methyl-5-(1-methyl-1H-pyrrol-2-yl)-phenyl]-benzenesulfonamide |
| 227 | 4-Methoxy-N-methyl-N-[2-methyl-5-(1-methyl-1H-pyrrol-2-yl)-phenyl]- |
| | benzenesulfonamide |
| 228 | 4-Methyl-N-[3-methyl-5-(1-methyl-1H-pyrrol-3-yl)-phenyl]-benzenesulfonamide |
| 229 | 4-Methoxy-N-methyl-N-[2-methyl-5-(1-methyl-1H-pyrrol-3-yl)-phenyl]- |
| | benzenesulfonamide |
| 230 | 4,N-Dimethyl-N-[2-methyl-5-(1-methyl-1H-pyrrol-2-yl)-phenyl]- |
| | benzenesulfonamide |
| 231 | 4-Methoxy-N-methyl-N-[3-methyl-5-(1-methyl-1H-pyrrol-2-yl)-phenyl]- |
| | benzenesulfonamide |
| 232 | 4,N-Dimethyl-N-[3-methyl-5-(1-methyl-1H-pyrrol-3-yl)-phenyl]- |
| | benzenesulfonamide |
| 233 | 4-Methoxy-N-[3-methyl-5-(1-methyl-1H-pyrrol-3-yl)-phenyl]-benzenesulfonamide |
| 234 | 4-Amino-N-[2-methyl-5-(1-methyl-1H-pyrrol-2-yl)-phenyl]-benzenesulfonamide |
| 235 | 4-Hydroxy-N-[2-methyl-5-(1-methyl-1H-pyrrol-2-yl)-phenyl]-benzenesulfonamide |
| 236 | 4-Chloro-N-[2-methyl-5-(1-methyl-1H-pyrrol-2-yl)-phenyl]-benzenesulfonamide |
| 237 | 4-Chloro-N-[2-methyl-5-(1-methyl-1H-pyrrol-3-yl)-phenyl]-benzenesulfonamide |
| 238 | 4-Hydroxy-N-[2-methyl-5-(1-methyl-1H-pyrrol-3-yl)-phenyl]-benzenesulfonamide |
| 239 | 4-Amino-N-[2-methyl-5-(1-methyl-1H-pyrrol-3-yl)-phenyl]-benzenesulfonamide |
| 240 | N-[5-(1,3-Dimethyl-1H-pyrrol-2-yl)-2-methyl-phenyl]-4-methyl-benzenesulfonamide |
| 241 | N-[5-(1,3-Dimethyl-1H-pyrrol-2-yl)-2-methyl-phenyl]-4-methoxy- |
| | benzenesulfonamide |
| 242 | N-[5-(1,4-Dimethyl-1H-pyrrol-3-yl)-2-methyl-phenyl]-4-methyl-benzenesulfonamide |
| 243 | N-[5-(1,4-Dimethyl-1H-pyrrol-3-yl)-2-methyl-phenyl]-4-methoxy- |
| | benzenesulfonamide |
| 244 | N-[3-(1,3-Dimethyl-1H-pyrrol-2-yl)-5-methyl-phenyl]-4-methyl-benzenesulfonamide |
| 245 | N-[5-(1,3-Dimethyl-1H-pyrrol-2-yl)-2-methyl-phenyl]-4-methoxy-N-methyl- |
| | benzenesulfonamide |
| 246 | N-[3-(1,4-Dimethyl-1H-pyrrol-3-yl)-5-methyl-phenyl]-4-methyl-benzenesulfonamide |
| 247 | N-[5-(1,4-Dimethyl-1H-pyrrol-3-yl)-2-methyl-phenyl]-4-methoxy-N-methyl- |
| | benzenesulfonamide |
| 248 | N-[5-(1,3-Dimethyl-1H-pyrrol-2-yl)-2-methyl-phenyl]-4,N-dimethyl- |
| | benzenesulfonamide |
| 249 | N-[3-(1,3-Dimethyl-1H-pyrrol-2-yl)-5-methyl-phenyl]-4-methoxy-N-methyl- |
| | benzenesulfonamide |
| 250 | N-[3-(1,4-Dimethyl-1H-pyrrol-3-yl)-5-methyl-phenyl]-4,N-dimethyl- |
| | benzenesulfonamide |
| 251 | N-[3-(1,4-Dimethyl-1H-pyrrol-3-yl)-5-methyl-phenyl]-4-methoxy- |
| | benzenesulfonamide |
| 252 | 4-Hydroxy-N-[2-methyl-5-(5-methyl-furan-2-yl)-phenyl]-benzenesulfonamide |
| 253 | 4-Amino-N-[2-methyl-5-(5-methyl-furan-2-yl)-phenyl]-benzenesulfonamide |
| 254 | 4-Chloro-N-[2-methyl-5-(5-methyl-furan-2-yl)-phenyl]-benzenesulfonamide |
| 255 | 4-Hydroxy-N-[2-methyl-5-(5-methyl-thiophen-2-yl)-phenyl]-benzenesulfonamide |
| 256 | 4-Amino-N-[2-methyl-5-(5-methyl-thiophen-2-yl)-phenyl]-benzenesulfonamide |
| 257 | 4-Chloro-N-[2-methyl-5-(5-methyl-thiophen-2-yl)-phenyl]-benzenesulfonamide |
| 258 | N-(5-Furan-2-yl-2-methyl-phenyl)-4-hydroxy-benzenesulfonamide |
| 259 | 4-Amino-N-(5-furan-2-yl-2-methyl-phenyl)-benzenesulfonamide |
| 260 | 4-Chloro-N-(5-furan-2-yl-2-methyl-phenyl)-benzenesulfonamide |
| 261 | 4-Hydroxy-N-(2-methyl-5-thiophen-2-yl-phenyl)-benzenesulfonamide |
| 262 | 4-Amino-N-(2-methyl-5-thiophen-2-yl-phenyl)-benzenesulfonamide |
| 263 | 4-Chloro-N-(2-methyl-5-thiophen-2-yl-phenyl)-benzenesulfonamide |
| 264 | 4-Hydroxy-N-(2-methyl-5-thiophen-3-yl-phenyl)-benzenesulfonamide |
| 265 | 4-Amino-N-(2-methyl-5-thiophen-3-yl-phenyl)-benzenesulfonamide |
| 266 | 4-Chloro-N-(2-methyl-5-thiophen-3-yl-phenyl)-benzenesulfonamide |
| 267 | N-(5-Furan-3-yl-2-methyl-phenyl)-4-hydroxy-benzenesulfonamide |
| 268 | 4-Amino-N-(5-furan-3-yl-2-methyl-phenyl)-benzenesulfonamide |
| 269 | 4-Chloro-N-(5-furan-3-yl-2-methyl-phenyl)-benzenesulfonamide |
| 270 | 4-Hydroxy-N-methyl-N-[2-methyl-5-(5-methyl-furan-2-yl)-phenyl]- |
| | benzenesulfonamide |
| 271 | 4-Amino-N-methyl-N-[2-methyl-5-(5-methyl-furan-2-yl)-phenyl]- |
| | benzenesulfonamide |
| 272 | 4-Chloro-N-methyl-N-[2-methyl-5-(5-methyl-furan-2-yl)-phenyl]- |
| | benzenesulfonamide |
| 273 | 4-Hydroxy-N-methyl-N-[2-methyl-5-(5-methyl-thiophen-2-yl)-phenyl]- |
| | benzenesulfonamide |
| 274 | 4-Amino-N-methyl-N-[2-methyl-5-(5-methyl-thiophen-2-yl)-phenyl]- |
| | benzenesulfonamide |
| 275 | 4-Chloro-N-methyl-N-[2-methyl-5-(5-methyl-thiophen-2-yl)-phenyl]- |
| | benzenesulfonamide |
| 276 | N-(5-Furan-2-yl-2-methyl-phenyl)-4-hydroxy-N-methyl-benzenesulfonamide |
| 277 | 4-Amino-N-(5-furan-2-yl-2-methyl-phenyl)-N-methyl-benzenesulfonamide |
| 278 | 4-Chloro-N-(5-furan-2-yl-2-methyl-phenyl)-N-methyl-benzenesulfonamide |
| 279 | 4-Hydroxy-N-methyl-N-(2-methyl-5-thiophen-2-yl-phenyl)-benzenesulfonamide |
| 280 | 4-Amino-N-methyl-N-(2-methyl-5-thiophen-2-yl-phenyl)-benzenesulfonamide |
| 281 | 4-Chloro-N-methyl-N-(2-methyl-5-thiophen-2-yl-phenyl)-benzenesulfonamide |
| 282 | 4-Hydroxy-N-methyl-N-(2-methyl-5-thiophen-3-yl-phenyl)-benzenesulfonamide |
| 283 | 4-Amino-N-methyl-N-(2-methyl-5-thiophen-3-yl-phenyl)-benzenesulfonamide |
| 284 | 4-Chloro-N-methyl-N-(2-methyl-5-thiophen-3-yl-phenyl)-benzenesulfonamide |
| 285 | N-(5-Furan-3-yl-2-methyl-phenyl)-4-hydroxy-N-methyl-benzenesulfonamide |
| 286 | 4-Amino-N-(5-furan-3-yl-2-methyl-phenyl)-N-methyl-benzenesulfonamide |
| 287 | 4-Chloro-N-(5-furan-3-yl-2-methyl-phenyl)-N-methyl-benzenesulfonamide |
| 288 | N-[5-(5-Cyano-furan-2-yl)-2-methyl-phenyl]-4-hydroxy-N-methyl- |
| | benzenesulfonamide |
| 289 | 4-Amino-N-[5-(5-cyano-furan-2-yl)-2-methyl-phenyl]-N-methyl- |
| | benzenesulfonamide |
| 290 | 4-Chloro-N-[5-(5-cyano-furan-2-yl)-2-methyl-phenyl]-N-methyl- |
| | benzenesulfonamide |
| 291 | 4-Hydroxy-N-methyl-N-[2-methyl-5-(5-nitro-furan-2-yl)-phenyl]- |
| | benzenesulfonamide |
| 292 | 4-Amino-N-methyl-N-[2-methyl-5-(5-nitro-furan-2-yl)-phenyl]-benzenesulfonamide |
| 293 | 4-Chloro-N-methyl-N-[2-methyl-5-(5-nitro-furan-2-yl)-phenyl]-benzenesulfonamide |
| 294 | N-[5-(5-Cyano-thiophen-3-yl)-2-methyl-phenyl]-4-hydroxy-benzenesulfonamide |
| 295 | 4-Amino-N-[5-(5-cyano-thiophen-3-yl)-2-methyl-phenyl]-benzenesulfonamide |
| 296 | 4-Chloro-N-[5-(5-cyano-thiophen-3-yl)-2-methyl-phenyl]-benzenesulfonamide |
| 297 | N-[5-(5-Cyano-furan-2-yl)-2-methyl-phenyl]-N-methyl-4-trifluoromethoxy- |
| | benzenesulfonamide |
| 298 | N-[5-(5-Cyano-furan-2-yl)-2-methyl-phenyl]-N-methyl-4-trifluoromethyl- |
| | benzenesulfonamide |
| 299 | N-[5-(5-Cyano-thiophen-3-yl)-2-methyl-phenyl]-4-trifluoromethoxy- |
| | benzenesulfonamide |
| 300 | N-[5-(5-Cyano-thiophen-3-yl)-2-methyl-phenyl]-4-trifluoromethyl- |
| | benzenesulfonamide |
| 301 | N-[5-(5-Cyano-furan-2-yl)-2-methyl-phenyl]-4-trifluoromethoxy- |
| | benzenesulfonamide |
| 302 | N-[5-(5-Cyano-furan-2-yl)-2-methyl-phenyl]-4-trifluoromethyl-benzenesulfonamide |
| 303 | N-[5-(2,4-Dimethyl-thiazol-5-yl)-2-methyl-phenyl]-4-hydroxy-benzenesulfonamide |
| 304 | N-[5-(3,5-Dimethyl-isoxazol-4-yl)-2-methyl-phenyl]-4-hydroxy-benzenesulfonamide |
| 305 | 4-Amino-N-[5-(2,4-dimethyl-thiazol-5-yl)-2-methyl-phenyl]-benzenesulfonamide |
| 306 | 4-Chloro-N-[5-(2,4-dimethyl-thiazol-5-yl)-2-methyl-phenyl]-benzenesulfonamide |
| 307 | 4-Amino-N-[5-(3,5-dimethyl-isoxazol-4-yl)-2-methyl-phenyl]-benzenesulfonamide |
| 308 | 4-Chloro-N-[5-(3,5-dimethyl-isoxazol-4-yl)-2-methyl-phenyl]-benzenesulfonamide |
| 309 | 4-Hydroxy-N-(2-methyl-5-oxazol-5-yl-phenyl)-benzenesulfonamide |
| 310 | 4-Amino-N-(2-methyl-5-oxazol-5-yl-phenyl)-benzenesulfonamide |
| 311 | 4-Chloro-N-(2-methyl-5-oxazol-5-yl-phenyl)-benzenesulfonamide |
| 312 | 4-Hydroxy-N-(2-methyl-5-pyrrol-1-yl-phenyl)-benzenesulfonamide |
| 313 | 4-Amino-N-(2-methyl-5-pyrrol-1-yl-phenyl)-benzenesulfonamide |
| 314 | 4-Chloro-N-(2-methyl-5-pyrrol-1-yl-phenyl)-benzenesulfonamide |
| 315 | 4-Hydroxy-N-[2-methyl-5-(3-methyl-pyrazol-1-yl)-phenyl]-benzenesulfonamide |
| 316 | 4-Amino-N-[2-methyl-5-(3-methyl-pyrazol-1-yl)-phenyl]-benzenesulfonamide |
| 317 | 4-Chloro-N-[2-methyl-5-(3-methyl-pyrazol-1-yl)-phenyl]-benzenesulfonamide |
| 318 | 4-Hydroxy-N-[2-methyl-5-(2-methyl-pyrrol-1-yl)-phenyl]-benzenesulfonamide |
| 319 | 4-Amino-N-[2-methyl-5-(2-methyl-pyrrol-1-yl)-phenyl]-benzenesulfonamide |
| 320 | 4-Chloro-N-[2-methyl-5-(2-methyl-pyrrol-1-yl)-phenyl]-benzenesulfonamide |
| 321 | N-(4,2′-Dimethyl-biphenyl-3-yl)-4-hydroxy-benzenesulfonamide |
| 322 | 4-Amino-N-(4,2′-dimethyl-biphenyl-3-yl)-benzenesulfonamide |
| 323 | 4-Chloro-N-(4,2′-dimethyl-biphenyl-3-yl)-benzenesulfonamide |
| 324 | N-(4,3′-Dimethyl-biphenyl-3-yl)-4-hydroxy-benzenesulfonamide |
| 325 | 4-Amino-N-(4,3′-dimethyl-biphenyl-3-yl)-benzenesulfonamide |
| 326 | 4-Chloro-N-(4,3′-dimethyl-biphenyl-3-yl)-benzenesulfonamide |
| 327 | N-(4,4′-Dimethyl-biphenyl-3-yl)-4-hydroxy-benzenesulfonamide |
| 328 | 4-Amino-N-(4,4′-dimethyl-biphenyl-3-yl)-benzenesulfonamide |
| 329 | 4-Chloro-N-(4,4′-dimethyl-biphenyl-3-yl)-benzenesulfonamide |
| 330 | 4-Hydroxy-N-(2-methyl-5-pyridin-2-yl-phenyl)-benzenesulfonamide |
| 331 | 4-Amino-N-(2-methyl-5-pyridin-2-yl-phenyl)-benzenesulfonamide |
| 332 | 4-Chloro-N-(2-methyl-5-pyridin-2-yl-phenyl)-benzenesulfonamide |
| 333 | 4-Hydroxy-N-(2-methyl-5-pyridin-3-yl-phenyl)-benzenesulfonamide |
| 334 | 4-Amino-N-(2-methyl-5-pyridin-3-yl-phenyl)-benzenesulfonamide |
| 335 | 4-Chloro-N-(2-methyl-5-pyridin-3-yl-phenyl)-benzenesulfonamide |
| 336 | 4-Hydroxy-N-(2-methyl-5-pyridin-4-yl-phenyl)-benzenesulfonamide |
| 337 | 4-Amino-N-(2-methyl-5-pyridin-4-yl-phenyl)-benzenesulfonamide |
| 338 | 4-Chloro-N-(2-methyl-5-pyridin-4-yl-phenyl)-benzenesulfonamide |
| 339 | N-[5-(5-Cyano-pyridin-3-yl)-2-methyl-phenyl]-4-hydroxy-benzenesulfonamide |
| 340 | 4-Amino-N-[5-(5-cyano-pyridin-3-yl)-2-methyl-phenyl]-benzenesulfonamide |
| 341 | 4-Chloro-N-[5-(5-cyano-pyridin-3-yl)-2-methyl-phenyl]-benzenesulfonamide |
| 342 | 4-Hydroxy-N-[2-methyl-5-(6-methyl-pyridin-2-yl)-phenyl]-benzenesulfonamide |
| 343 | 4-Amino-N-[2-methyl-5-(6-methyl-pyridin-2-yl)-phenyl]-benzenesulfonamide |
| 344 | 4-Chloro-N-[2-methyl-5-(6-methyl-pyridin-2-yl)-phenyl]-benzenesulfonamide |
| 345 | N-[5-(5-Cyano-pyridin-2-yl)-2-methyl-phenyl]-4-hydroxy-benzenesulfonamide |
| 346 | 4-Amino-N-[5-(5-cyano-pyridin-2-yl)-2-methyl-phenyl]-benzenesulfonamide |
| 347 | 4-Chloro-N-[5-(5-cyano-pyridin-2-yl)-2-methyl-phenyl]-benzenesulfonamide |
| 348 | N-[5-(5-Cyano-pyridin-2-yl)-2-methyl-phenyl]-4-trifluoromethoxy- |
| | benzenesulfonamide |
| 349 | N-[5-(5-Cyano-pyridin-2-yl)-2-methyl-phenyl]-4-trifluoromethyl- |
| | benzenesulfonamide |
| 350 | 4-Hydroxy-N-[5-(5-methoxy-pyridin-2-yl)-2-methyl-phenyl]-benzenesulfonamide |
| 351 | 4-Amino-N-[5-(5-methoxy-pyridin-2-yl)-2-methyl-phenyl]-benzenesulfonamide |
| 352 | 4-Chloro-N-[5-(5-methoxy-pyridin-2-yl)-2-methyl-phenyl]-benzenesulfonamide |
| 353 | N-[5-(5-Methoxy-pyridin-2-yl)-2-methyl-phenyl]-4-trifluoromethoxy- |
| | benzenesulfonamide |
| 354 | N-[5-(5-Methoxy-pyridin-2-yl)-2-methyl-phenyl]-4-trifluoromethyl- |
| | benzenesulfonamide |
| 355 | N-[5-(5-Fluoro-pyridin-2-yl)-2-methyl-phenyl]-4-hydroxy-benzenesulfonamide |
| 356 | 4-Amino-N-[5-(5-fluoro-pyridin-2-yl)-2-methyl-phenyl]-benzenesulfonamide |
| 357 | 4-Chloro-N-[5-(5-fluoro-pyridin-2-yl)-2-methyl-phenyl]-benzenesulfonamide |
| 358 | N-[5-(5-Fluoro-pyridin-2-yl)-2-methyl-phenyl]-4-trifluoromethoxy- |
| | benzenesulfonamide |
| 359 | N-[5-(5-Fluoro-pyridin-2-yl)-2-methyl-phenyl]-4-trifluoromethyl- |
| | benzenesulfonamide |
| 360 | 4-Hydroxy-N-[2-methyl-5-(5-nitro-pyridin-2-yl)-phenyl]-benzenesulfonamide |
| 361 | 4-Amino-N-[2-methyl-5-(5-nitro-pyridin-2-yl)-phenyl]-benzenesulfonamide |
| 362 | 4-Chloro-N-[2-methyl-5-(5-nitro-pyridin-2-yl)-phenyl]-benzenesulfonamide |
| 363 | N-[2-Methyl-5-(5-nitro-pyridin-2-yl)-phenyl]-4-trifluoromethoxy- |
| | benzenesulfonamide |
| 364 | N-[2-Methyl-5-(5-nitro-pyridin-2-yl)-phenyl]-4-trifluoromethyl-benzenesulfonamide |
| 365 | 4-Amino-N-(4′-cyano-4-methyl-biphenyl-3-yl)-benzenesulfonamide |
| 366 | N-[5-(6-Cyano-pyridin-3-yl)-2-methyl-phenyl]-4-hydroxy-benzenesulfonamide |
| 367 | 4-Amino-N-[5-(6-cyano-pyridin-3-yl)-2-methyl-phenyl]-benzenesulfonamide |
| 368 | 4-Chloro-N-[5-(6-cyano-pyridin-3-yl)-2-methyl-phenyl]-benzenesulfonamide |
| 369 | N-(3′,4′-Dicyano-4-methyl-biphenyl-3-yl)-4-hydroxy-benzenesulfonamide |
| 370 | 4-Amino-N-(3′,4′-dicyano-4-methyl-biphenyl-3-yl)-benzenesulfonamide |
| 371 | 4-Chloro-N-(3′,4′-dicyano-4-methyl-biphenyl-3-yl)-benzenesulfonamide |
| 372 | N-(4′-Cyano-3′-fluoro-4-methyl-biphenyl-3-yl)-4-hydroxy-benzenesulfonamide |
| 373 | 4-Amino-N-(4′-cyano-3′-fluoro-4-methyl-biphenyl-3-yl)-benzenesulfonamide |
| 374 | 4-Chloro-N-(4′-cyano-3′-fluoro-4-methyl-biphenyl-3-yl)-benzenesulfonamide |
| 375 | N-(4′-Cyano-4,3′-dimethyl-biphenyl-3-yl)-4-hydroxy-benzenesulfonamide |
| 376 | 4-Amino-N-(4′-cyano-4,3′-dimethyl-biphenyl-3-yl)-benzenesulfonamide |
| 377 | 4-Chloro-N-(4′-cyano-4,3′-dimethyl-biphenyl-3-yl)-benzenesulfonamide |
| 378 | 3-Fluoro-N-(5-furan-2-yl-2-methyl-phenyl)-4-methyl-benzenesulfonamide |
| 379 | 3-Fluoro-N-(5-furan-2-yl-2-methyl-phenyl)-4-methoxy-benzenesulfonamide |
| 380 | 3-Fluoro-4-methyl-N-[2-methyl-5-(5-methyl-furan-2-yl)-phenyl]- |
| | benzenesulfonamide |
| 381 | 3-Fluoro-4-methoxy-N-[2-methyl-5-(5-methyl-furan-2-yl)-phenyl]- |
| | benzenesulfonamide |
| 382 | N-(4,4′-Dimethyl-biphenyl-3-yl)-3-fluoro-4-methyl-benzenesulfonamide |
| 383 | N-(4,4′-Dimethyl-biphenyl-3-yl)-3-fluoro-4-methoxy-benzenesulfonamide |
| 384 | 3-Fluoro-N-[5-(5-methoxy-pyridin-2-yl)-2-methyl-phenyl]-4-methyl- |
| | benzenesulfonamide |
| 385 | 3-Fluoro-4-methoxy-N-[5-(5-methoxy-pyridin-2-yl)-2-methyl-phenyl]- |
| | benzenesulfonamide |
| 386 | 3-Fluoro-4-methyl-N-(2-methyl-5-pyridin-3-yl-phenyl)-benzenesulfonamide |
| 387 | 3-Fluoro-4-methoxy-N-(2-methyl-5-pyridin-3-yl-phenyl)-benzenesulfonamide |
| 388 | N-[5-(4-Cyano-thiophen-2-yl)-2-methyl-phenyl]-3-fluoro-4-methyl- |
| | benzenesulfonamide |
| 389 | N-[5-(4-Cyano-thiophen-2-yl)-2-methyl-phenyl]-3-fluoro-4-methoxy- |
| | benzenesulfonamide |
| 390 | N-[5-(5-Cyano-thiophen-3-yl)-2-methyl-phenyl]-3-fluoro-4-methoxy- |
| | benzenesulfonamide |

### Discussion

It follows directly from the data shown in the examples above that the compounds of the invention have the common property of inhibiting HIF function and inhibiting cell proliferation. Thus, these compounds are useful as therapeutic compounds for the treatment or prevention of a disease or disorder selected from the group consisting of an inflammatory disease, a hyperproliferative disease or disorder, a hypoxia-related pathology and a disease characterized by excessive vascularisation.

In addition to the above described useful properties that are shared by all compounds of the invention, these compounds also exhibit different degrees of potency with respect to inhibiting tubulin organization. This shows a further valuable contribution of this invention which lies in the provision of therapeutic compounds that can be administered selectively on a case to case basis to patients depending on their disease state, constitution and genetic predisposition. In this context it is further possible to decide if the patient is to be treated with compounds of the invention that do or do not possess an additional activity as tubulin inhibitors.

1. A compound having a structure according to formula I: 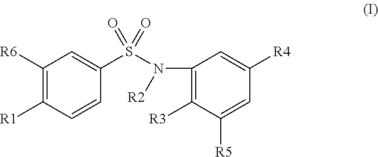
wherein
R¹ is selected from a group consisting of H, alkyl, alkenyl, alkynyl, —CN, halogen, —OH, alkoxy, —SH, S-alkyl, —NH₂, NH-alkyl, N-bis-alkyl, NHOH, NMeOH, NMe(OMe), —NO₂, —CF₃, —OCF₃ and C₁-C₄ hydroxyalkyl.
R² is H or C₁-C₄ alkyl;
R³ is H or —CH₃;
R⁴ is phenyl or monocyclic 5- or 6-membered heteroaryl; optionally substituted with one or more substituents selected from the group consisting of:
alkyl, alkenyl, alkynyl, alkoxy, halogen, —CN, —CF₃, —OCF₃, C₁-C₄ hydroxyalkyl, —OH, —SH, S-alkyl, —CN, N-bis-alkyl, cyanoacetylene, —NO₂, —NR⁷R⁸, —C(O)R²⁰, N—O (wherein the nitrogen atom is integral part of the monocyclic 5- or 6-membered heteroaryl) and two substituents which form together a dioxymethylene bridge (—O—CH₂—O—);
R⁵ is H or —CH₃;
R⁶ is selected from the group consisting of H, halogen, alkyl, alkoxy, alkenyl, alkynyl, S-alkyl, —OH, —NR⁷R⁸, —CN, N-bis-alkyl, —SH, —CF₃ and —OCF₃;
or R⁶ forms together with R¹ a dioxymethylene bridge (—O—CH₂—O—);
R⁷ is H or alkyl;
R⁸ is H or C₁-C₄ alkyl; and
R²⁰ is C₁-C₄ alkyl;
with the proviso that R⁴ is not 3-alkoxy-pyridazine-5-yl; that if R⁴ is phenyl, then the 2- and 5-position of the phenyl ring may not be substituted with two methoxy substituents at the same time; and that R³ and R⁵ are not at the same time H.

2. The compound according to claim 1, wherein the compound has a structure according to formula II:

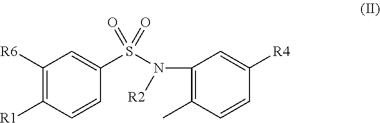

3. The compound according to claim 1, wherein the compound has a structure according to formula III:

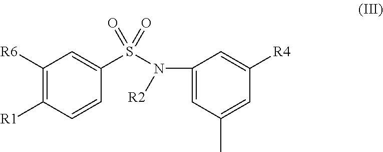

4. The compound according to claim 1, wherein R⁴ has a structure according to formula IV: 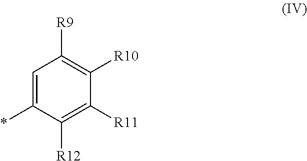
wherein
R⁹ and R¹⁰ are each individually selected from the group consisting of H, alkyl, C₁-C₄ alkenyl, alkynyl, —CN, —C(O)R²⁰, cyanoacetylene, halogen, —OH, C₁-C₄ alkoxy, —SH, C₁-C₄ S-alkyl, —NH₂, C₁-C₄ NH-alkyl, C₁-C₄ N-bis-alkyl, —NO₂, —CF₃, —OCF₃, and C₁-C₄ hydroxyalkyl,
or R⁹ and R¹⁰ form together a dioxymethylene bridge (—O—CH₂—O—);
R¹¹ and R¹² are each individually selected from the group consisting of H, C₁-C₄ alkyl, C₁-C₄ alkenyl, C₁-C₄ alkynyl, —CN, halogen, —OH, alkoxy, —SH, C₁-C₄ S-alkyl, —CF₃, —OCF₃, —NH₂, —N(CH₃)₂ and C₁-C₄ hydroxyalkyl;
with the proviso that R⁹ and R¹² may not be methoxy at the same time;
R²⁰ has the above indicated meaning; and
* indicates the bond between R⁴ and the compound according to any of formulas (I) through (III).

5. The compound according to claim 4, wherein R¹¹ and R¹² are H.

6. The compound according to claim 5, wherein
R³ is methyl;
R² is H, methyl or ethyl; and
R⁵ and R⁶ are H.

7. The compound according to claim 1, wherein R⁴ has a structure according to formula V: 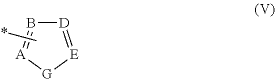
wherein
A, B, D and E are each individually selected from the group consisting of a nitrogen atom, CR¹³ and N—O;
G is selected from the group consisting of an oxygen atom, a sulphur atom and NR¹⁴;
R¹³ is selected from the group consisting of H, C₁-C₃ alkyl, C₁-C₃ alkoxy, —OH, —SH, —CF₃, —OCF₃, halogen, —NR¹⁵R¹⁶, —NO₂, —CN, —C(O)R²⁰, acetylene, cyanoacetylene, C₁-C₄ hydroxyalkyl and a σ (sigma) bond connecting R⁴ to the compound according to any of formulas (I) through (III); and
R¹⁴ is selected from the group consisting of H, C₁-C₄ alkyl and a σ (sigma) bond connecting R⁴ to the compound according to any of formulas (I) through (III); and
R¹⁵ and R¹⁶ are each individually either H or C₁-C₄ alkyl;
R²⁰ has the above indicated meaning; and
* indicates the bond between R⁴ and the compound according to any of formulas (I) through (III).

8. The compound according to claim 1, wherein R⁴ has a structure according to formula VI: 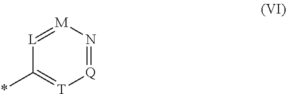
wherein
L and T are each individually either a CH group or a nitrogen atom or N—O;
M, N and Q are each individually selected from the group consisting of a nitrogen atom, a CR¹⁷ group and N—O;
R¹⁷ is selected from the group consisting of H, C₁-C₃ alkyl, C₁-C₃ alkoxy, —CF₃, —OCF₃, halogen, —OH, —NO₂, —SH, C₁-C₃ S-alkyl, —NR¹⁵R¹⁶, C₁-C₄ hydroxyalkyl, —C(O)R²⁰, acetylene, cyanoacetylene and —CN;
R¹⁵ and R¹⁶ are each individually either H or C₁-C₄ alkyl;
R²⁰ has the above indicated meaning; and
* indicates the bond between R⁴ and the compound according to any of formulas (I) through (III).

9. The compound according to claim 1, wherein R⁴ is selected from the group consisting of: 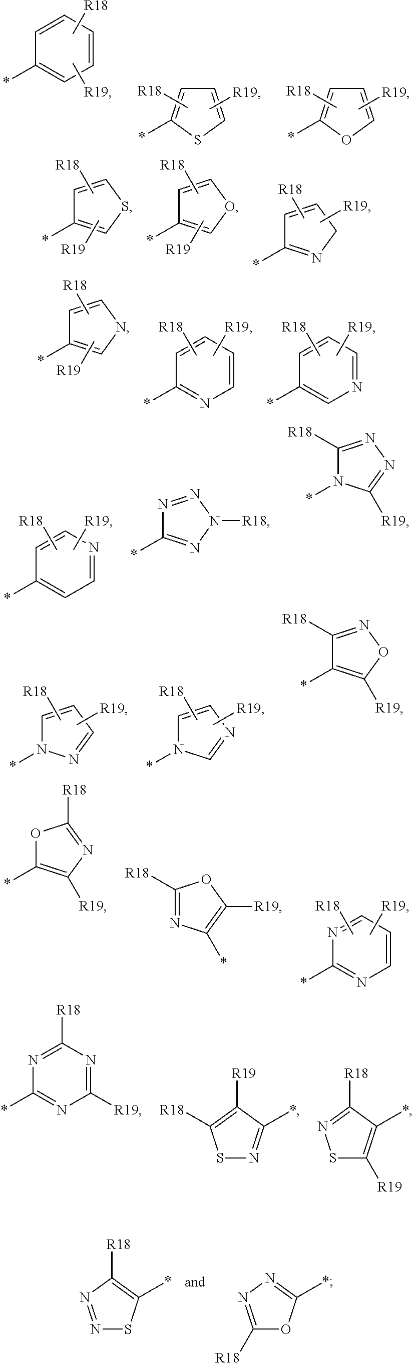
wherein R¹⁸ and R¹⁹ are each individually selected from the group consisting of H, C₁-C₃ alkyl, C₁-C₃ alkoxy, —CF₃, —OCF₃, halogen, —OH, —NO₂, —SH, C₁-C₃ S-alkyl, —NR¹⁵R¹⁶, C₁-C₄ hydroxyalkyl, alkynyl, alkenyl, —C(O)R²⁰, cyanoacetylene and —CN;
and R¹⁵, R¹⁶ and R²⁰ have the above indicated meaning.

10. The compound according to claim 1, wherein R² is H and R⁴ is selected from the group consisting of: 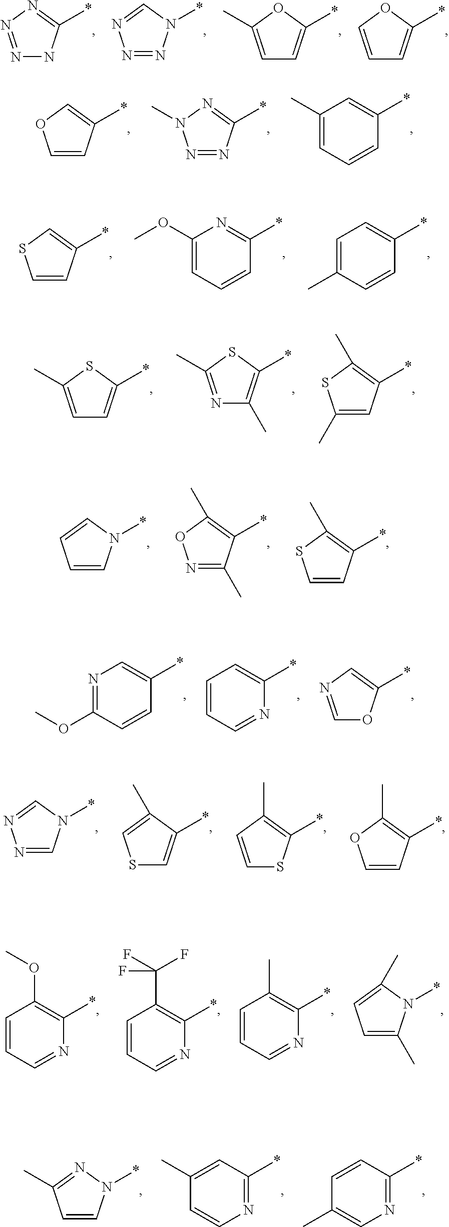 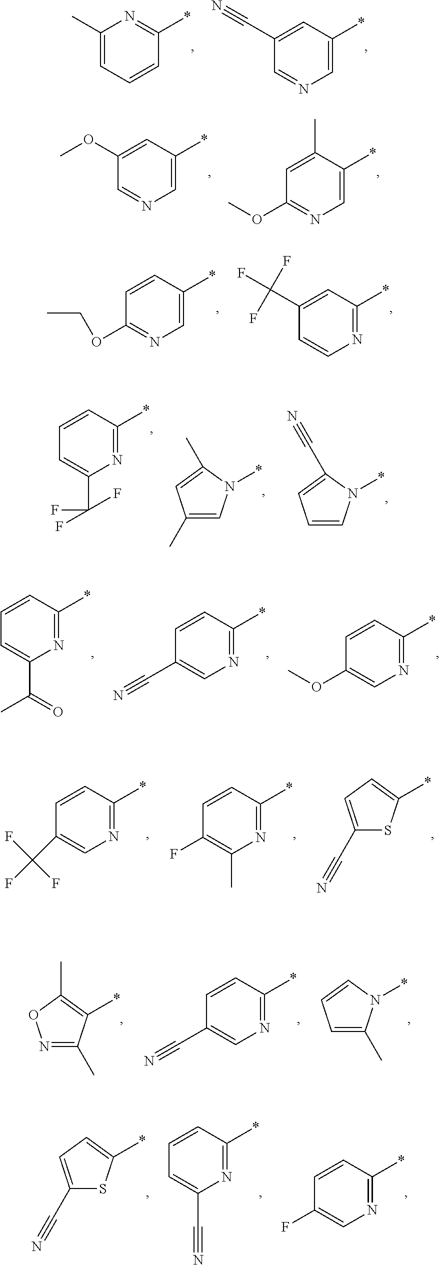 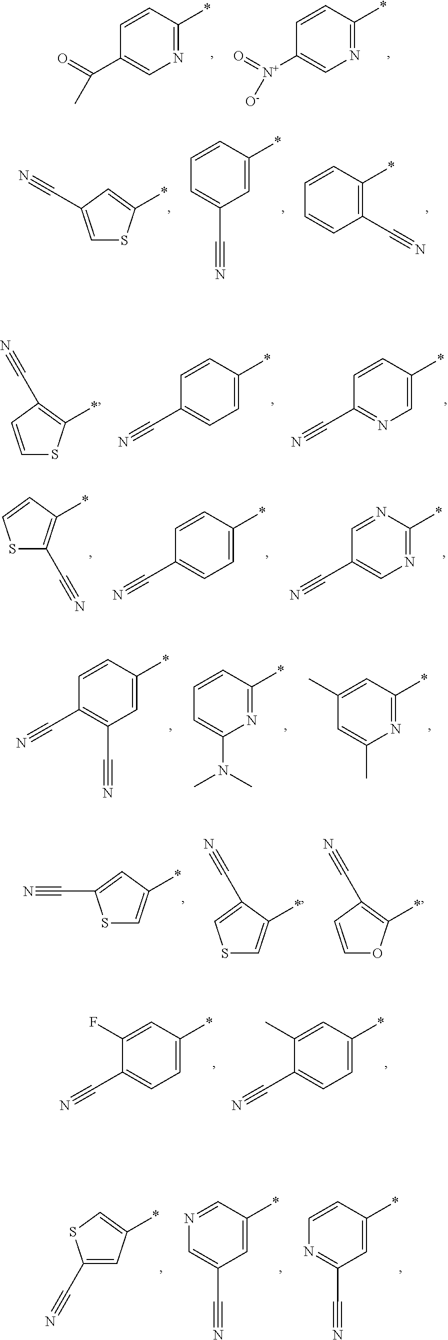 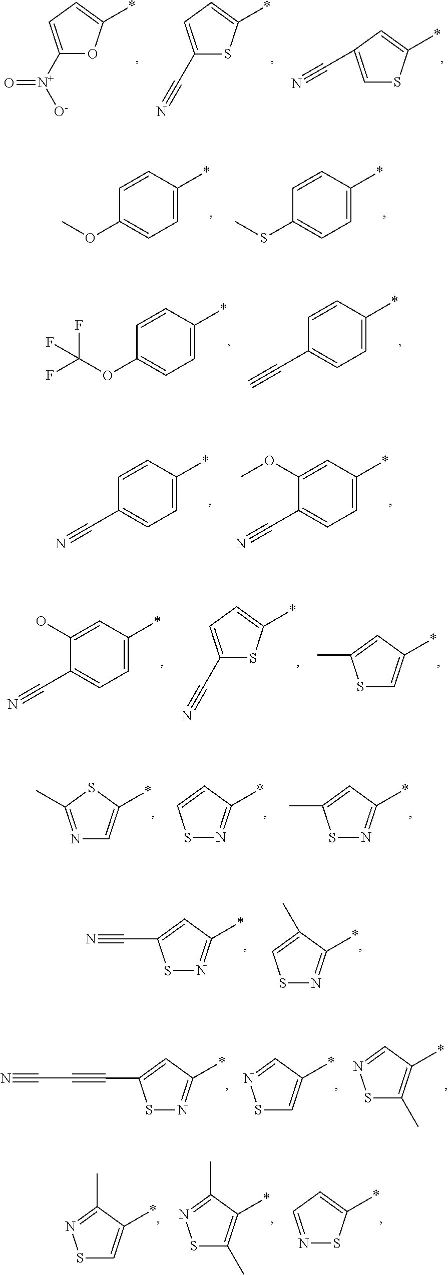 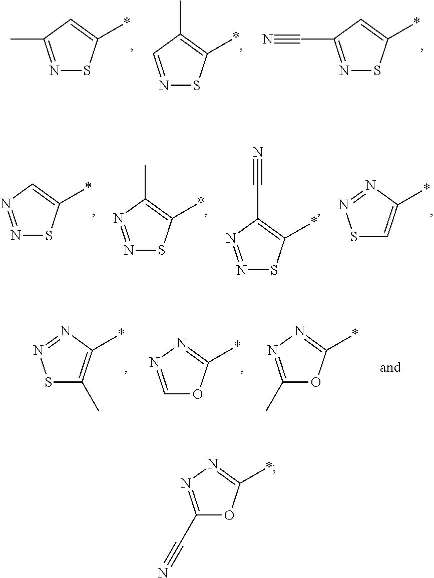
wherein * indicates the bond between R⁴ and the compound according to any of formulas (I) through (III).

11. The compound according to claim 1, wherein R² and/or R⁶ is H.

12. A compound according to claim 1 or pharmaceutically acceptable salt thereof, for the prevention or treatment of a disease or disorder.

13. A pharmaceutical composition comprising a compound according to claim 1 or pharmaceutically acceptable salt thereof and a second therapeutic agent useful for the treatment or prevention of a disease or disorder selected from the group consisting of an inflammatory disease, a hyperproliferative disease or disorder, a hypoxia related pathology and a disease characterized by pathophysiological hyper-vascularisation, and, optionally, a pharmaceutically acceptable carrier or excipient.

14. A method of preparing a medicament, comprising the steps of:
providing a compound according to claim 1, or a pharmaceutically acceptable salt thereof; and
using said compound for the preparation of a medicament for the treatment or prevention of a disease or disorder selected from the group consisting of an inflammatory disease, a hyperproliferative disease or disorder, a hypoxia related pathology and a disease characterized by pathophysiological hyper-vascularisation.

15. The method according to claim 14, wherein the inflammatory disease is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichtyosises; alopecia greata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia greata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and periorale dermatitis.

16. The method according to claim 14, wherein the hyperproliferative disease is selected from the group consisting of a tumor or cancer disease, precancerosis, dysplasia, histiocytosis, a vascular proliferative disease and a virus-induced proliferative disease.

17. The method according to claim 16, wherein the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

18. A method for treating a hyperproliferative disease or disorder, comprising the step of: administering a compound according to claim 1 or a pharmaceutically acceptable salt thereof to a patient prior to, during and/or after the patient was subjected to at least one therapy selected from the group consisting of: radiation therapy; chemotherapy; immunotherapy; laser/microwave thermotherapy; and gene therapy using antisense DNA and/or RNA.
